(19) United States
(12) Patent Application Publication
(10) Pub. No.: US 2003/0171729 A1
(43) Pub. Date: Sep. 11, 2003

(54) Multifunctional containment sheet and system for absorbent atricles

(76) Inventors: James Martin Kaun, Neenah, WI (US); Kambiz Bayat Makoui, Neenah, NC (US); Dean Michael Laux, Appleton, WI (US); Toan Thanh LeMinh, Greenville, WI (US); Brenda Marie Nelson, Appleton, WI (US); John Olszewski, Menasha, WI (US)

Correspondence Address: PAULEY PETERSEN KINNE & ERICKSON, 2800 WEST HIGGINS ROAD, SUITE 365, HOFFMAN ESTATES, IL, 60195, US

(21) Appl. No.: 10/032,805

(22) Filed: Dec. 28, 2001

(51) Int. Cl. A61F 13/20; A61F 13/15

(52) U.S. Cl. 604/382

(57) ABSTRACT

This invention relates to a containment tissue of an absorbent article such as a diaper, treated to have regions with hydrophobic properties to maximize the strength of the containment tissue when wet. The result is less superabsorbent material contacting the user and therefore less skin irritation. Additional benefits of this invention include enhanced leakage protection and reduced skin wetting, which also reduces skin irritation. A hydrophobic agent such as a sizing agent or adhesive is used to create zones of water resiliency on the containment tissue in various regions, patterns, and/or configurations. The treatments of the containment tissue are applied by known application methods such as gravure printing, spraying, or ink jet printing. Liquid insults easily wick through untreated containment tissue but stop at the boundaries of treated regions. This provides a method of leakage control when wicking is halted before the liquid reaches the edges of the absorbent article. Wet strength is also greatly increased as the hydrophobic treated regions retain substantially all the original dry strength.

### FIELD OF THE INVENTION

This invention relates to cover and containment sheets useful in absorbent articles such as diapers, training pants, adult incontinence garments, feminine care products, and medical devices. Cover and containment sheets provide a barrier and enclosure to prevent the contact and deposition of various absorbent components on the skin of the user. The improved containment sheet of this invention is chemically treated to increase dry and wet strength, control fluid absorbency through and within the sheet, lower rewet, improve air and vapor exchange, and produce a visual indicator.

### BACKGROUND OF THE INVENTION

Many personal care absorbent articles include a body-side liner (sometimes referred to as a liner, topsheet layer, or inner cover sheet), a containment sheet, an absorbent core, and some type of backing material (an outer cover) which is generally liquid impervious to help prevent leakage. The types of body-side liner materials generally fall into two main groups, either film or nonwoven body-side liners. The choice between film or nonwoven body-side liners depends on the performance, fit, and aesthetic attributes required of the product. The advantage of film body-side liners for sanitary napkins is that they provide a relatively clean and dry surface as fluid tends to pass through the film layer and into the interior of the absorbent product. A drawback, however, is that such film layers do not provide the degree of softness and comfort that a nonwoven body-side liner material can provide. An additional drawback is the smooth, slick, non-cloth-like feel that is characteristic of many films. Nonwoven-based body-side liner materials, on the other hand, are very soft and cloth-like in feel, but tend to retain more of the fluid at or just below the surface of the body-side liner material which, in turn, makes the product suffer from the standpoint of properties such as cleanliness and dryness.

Absorbent articles have typically employed various types of absorbent pads composed of cellulose fibers. Particular absorbent garments have been configured to control the distribution of absorbed liquid. For example, an absorbent article can have a liquid permeable transport layer which is located between a topsheet layer and an absorbent body. In other configurations, a conventional absorbent member can have fluid storage and acquisition zones composed of cellulosic fluff mixed with absorbent gelling particles, and may include a dual-layer absorbent core arrangement comprising a bottom fluff pad containing hydrogel particles, and a top fluff pad with little or no hydrogel particles.

In addition, the absorbent core may consist of synthetic fibers in combination with natural fibers. These types of structures tend to be more resilient and possess a more uniform pore structure under load or when in contact with fluid than traditional absorbents.

The purpose of a containment sheet, also sometimes referred to as a barrier sheet, is to provide a barrier to prevent the contact and deposition of various absorbent components on the skin of the user. In addition, the sheet may contribute to the integrity of the absorbent structure allowing more efficient intake, distribution and retention of the fluid.

An absorbent composite typically is comprised of loosely bonded fibers, highly absorbent polymeric and other materials. Various means are used to maintain the integrity and continuity of the structure to maximize the function and performance of the absorbent. Additives (e.g., binders, synthetic fibers) and processing (compaction and layering) are often employed to aid integrity.

However, the components or the absorbent materials often separate from the body of the absorbent pad during manufacture, handling, and/or use. Displacement or loss of these materials may result in a decrease in efficiency of the product and deposition of these materials on the user's skin or other surfaces. The product may not perform as well in terms of leakage, increase irritation to the skin, require more cleaning, and give a visible indication of poor product integrity. Fibers and absorbent polymers in contact with the skin may result in irritation due to the components themselves or accumulation and storage of fluid next to the skin. Gel beads from super absorbent polymers are an example of one category where containment is essential. Failure to do so will lead to the problems cited above.

A typical containment sheet will restrict the migration of large and small particles through the sheet and yet provide easy passage of liquids from the user to the absorbent material. Small pore size and hydrophilicity are the characteristics required for these functions. The containment sheet is also required to maintain its barrier properties throughout the use cycle of the product. Key properties controlling the permanence and integrity of the sheet include sufficient dry and wet strength to avoid rupture or tearing in use.

In addition, containment sheets preferably have certain properties to avoid negative impact on the primary intended purpose of the absorbent article while providing its containment function. Desired properties include high intake, the ability to desorb the liquid away from the user, the ability to minimize fluid flowback (rewet), the ability to maintain its integrity in both dry and wet environments, and the ability to allow the transmission of water vapor away from the wearer. Suitable containment sheets should not feel damp or clammy to the user or provider, possess adequate strength while being processed in manufacturing, comprise materials that are easily reclaimed and recycled, and provide the intended benefits at the lowest cost.

An additional desire is for the containment sheet to contribute to the performance of the absorbent article beyond that of the containment function. These multifunctional properties could aid absorbent article performance in terms of reduced leakage, improved skin health, and serving as a visual indicator for communicating various attributes or messages. The hydrophilic nature of a cellulose-based containment sheet, such as tissue, imparts several undesirable characteristics that are detrimental to the performance of absorbent articles. First, despite the use of a wet strength papermaking additive, cellulosic containment sheets typically loose between 80 to 85% of the original dry strength when wetted. The numerous stresses from body movement and swelling of the absorbent core will often exceed the wet strength and result in tearing of the containment tissue allowing superabsorbent particles and fiber to deposit onto the skin.

Second, cellulosic containment sheets have excellent wicking properties that allow the transport of liquid over the entire surface of the tissue including the edges of the product. This is desirable from an absorbency perspective but can increase potential leakage due to the containment sheet's proximity with the skin or outer edges of the absorbent article. Unimpeded fluid distribution increases the chances of leakage and therefore makes a need to control the extent and range of the wicking of the tissue. In the case of medical products, it is desirable to restrict the absorption of fluids to the interior of the article to minimize potential contact with fluids with the user, caregiver, clothing, or linen.

A totally hydrophilic containment material will impart an undesirable wet or clammy feel to the product. Wetting is desirable in the target area for the insult or discharge, but peripheral areas that are not involved in acquiring the fluid become needlessly wet due to the wicking properties of the material. It is desirable to minimize the wetted surface of the containment sheet to increase the comfort of the product.

Third, the hydrophilic nature of the containment sheet made with cellulosic fibers results in the retention of some liquid which will obstruct many of the pores within the sheet. The exchange of vapor and air to and away from the skin is thereby reduced which will negatively impact the condition of the skin. A need exists to preserve some of the original air and vapor permeability of the tissue to enhance breathability, a key factor to maintain skin health.

Synthetic materials, typically in the form of nonwovens and films, are often used as a cover material in many absorbent articles. Nonwovens can be treated to become hydrophilic to allow fluids to pass through in the z-direction. However, they have difficulty in wicking and spreading out fluid in the x-y plane. The hydrophilic nature can also be somewhat fugitive due to poor substantivity of economical surfactants. Pore size distribution is also problematic since the processes used to make economical nonwovens typically have relatively poor formation and thus a high incidence of undesirable large pores.

In some applications, the non-woven structure is designed to have low basis weight and large pore size to allow high rates of liquid penetration. However, liquid and absorbent material can easily be pushed through the large openings and onto the user. For example, many diapers and incontinent products currently marketed use an open cover or liner on the skin side. An underlying cellulosic tissue is required to create a barrier system to address the deficiencies of the synthetic liner in terms of containment of the absorbent material.

In the case of single layer cast apertured films there is generally a lack of any capability to wick fluid along the plane of the film. In addition, they are suitable for low flow applications such as menses containment.

What is needed is a multifunctional containment sheet for absorbent articles that provides improved properties to prevent the negative results of absorbent materials on the skin, ability to control fluid wicking and penetration, and higher vapor permeability to improve air and water vapor exchange within the absorbent article at the skin interface and acts as a visual indicator.

### SUMMARY OF THE INVENTION

An object of this invention is to improve the dry and wet strength of a containment sheet by providing a continuous network that supplements the original underlying dry strength or retains the original dry strength in the treated areas when wetted. The network is created by application of hydrophobic or hydrophilic chemistry and is patterned to minimize interference with the liquid handling properties of the sheet. Hydrophilic regions are of sufficient size and located to continue to handle the intake requirements of the product.

In one embodiment of this invention a cellulosic web is treated with a hydrophobic chemistry such as a “sizing agent” to create a continuous network or networks of hydrophobic regions for improved strength surrounding untreated areas to provide fluid intake. Applying “sizing” in a specific design, such as a grid pattern, results in greatly increased wet strength as the hydrophobic regions retain substantially all the original dry strength. One aspect of this invention is using a sizing agent to create regions of hydrophobicity on the containment tissue. A “sizing agent” is any chemical that imparts water repellency to cellulosic fibers. Various sizing agents are known in the paper-making art and are commonly added to control the penetration of aqueous liquids into paper materials such as butcher's wrap, milk cartons, liner boards, fine papers, and newsprint. Patterns of application can be determined by the needs of different types of absorbent articles. The treatments of the containment sheets are applied by known application methods such as gravure printing, flexographic printing, spraying, or inkjet printing. Preferred application methods result in uniform application in desired amounts and patterns. Printing a sizing agent onto a containment tissue creates treated regions of non-absorbency.

Another object of this invention is to modify and control the fluid handling properties of the containment sheet and advantageously limit the wicking of liquid in the x-y plane of the sheet and direct liquid flow through in the perpendicular direction to the plane (z-direction).

In one embodiment of this invention specified regions of the containment sheet are treated with a hydrophobic chemistry to prevent wicking of liquid to those areas. Liquid insults easily wick throughout the untreated areas but stop at the boundaries of the treated areas. The containment sheet is treated at specific problematic locations perhaps preferentially in the waist and leg areas to reduce leakage and rewetting of clothing. The function is analogous to that of impermeable flaps inserted inside waste band and leg areas of absorbent products.

Another embodiment of this invention resembles a window pane. The absorbent target area (the window) is untreated to accept the insults and the surrounding treated frame is hydrophobic to inhibit liquid run-off and wicking towards the edges of the product.

In another embodiment of this invention, such as a medical product, treatment of a peripheral area of a bandage or similar device will retard the wicking of the blood towards the edges of the article and minimize leakage and contact. The sheet will still retain absorption properties where needed.

Another object of this invention is to improve air and vapor exchange within the absorbent article thereby improving skin health and wound healing. Hydrophobic regions on the containment sheet retain at least substantially their original air and vapor permeability to assist in the passage of air and other therapeutic vapors into the absorbent article and unwanted vapors out of the absorbent article.

In one embodiment the hydrophobic treatment is applied in regions where ventilation is desirable yet in a defined pattern and surface area to maintain the necessary fluid intake and distribution. The hydrophobic regions allow this air exchange to occur while the surrounding areas are hydrophilic. The patterns can be made contiguous or as discrete zones and the breathable hydrophobic region can be varied depending on location within the absorbent article.

Another object of this invention is to prevent rewetting of skin when pressure is applied onto a partially saturated absorbent material. The presence of moisture at the skin interface is one mechanism that will damage the stratum corneum, the outer protective skin layer. Compression of the absorbent article during use can generate free liquids from the absorbent material and bring the absorbent interface in closer proximity to the skin. Removal of liquid and subsequent rewet prevention from areas in proximity to the skin is essential not only for leakage control but is vital in maintaining skin barrier protection.

In one embodiment, selective hydrophobic regions are placed in areas susceptible to skin irritation to minimize the contact of wetted sheet directly or adjacent to the skin (when used with a liner or cover stock). Any applied pressure to the saturated absorbent will not rewet the sheet nor penetrate through the sheet/liner system to the skin in the hydrophobic regions.

In another embodiment, a fully hydrophobic portion of the sheet is placed inside and adjacent to the breathable outer cover. This combination will resist penetration of liquids and minimize the perception of outer cover dampness. Outer cover dampness is a common consumer objection to diapers; in particular to those diapers that employ a breathable outer cover.

Another object of this invention is to provide a visual indicator that is seen after the product is wetted. The visual indicator is created by the contrast between the wetted and non-wetted regions of the sheet, and is beneficial for aesthetics reasons or to communicate messages concerning wetness or bringing attention to a product improvement. No chemical reaction is required to generate the contrast in appearance. The high permeability of the sheet will not restrict the intended breathability of the outer cover.

In one embodiment, a hydrophilic region with a distinctive pattern or text is located in an easy to view area such as the inside at the waistband or special window. This placement of the visual indicator is particularly useful to indicate wetness in newborns where it is difficult to tell whether an infant has wetted the diaper, due to the low volume. Unlike the high capacity absorbent core, the low capacity of the sheet allows the liquid to adequately saturate the hydrophilic portion of the display area with a minimum of fluid to achieve the desired visual contrast. In another embodiment the hydrophilic region is in the form of graphics, log, trademarks, or message (i.e., warning/instruction) that are visible when the product is wetted or removed.

In another embodiment, differing levels of contrast intensity are achieved between the wet and dry regions by adjusting the original color of the sheet. The intensity and thus the perception of the visual indicator can be subdued (e.g., white) or more intense to be easily seen (e.g., blue).

In another embodiment, of this invention any of the above embodiments can be incorporated into a multilayer system, typically including a synthetic liner and a cellulosic containment sheet with hydrophobic and hydrophilic regions as described previously. The multilayer system's resistance to liquid penetration in the hydrophobic regions while under pressure is typically substantially greater than either of the individual components alone. The multilayer system decreases the rewet under applied loads thus reducing leakage and skin rewetting.

### BRIEF DESCRIPTION OF THE DRAWINGS

FIG. 1 is an exploded perspective view of an absorbent article according to one embodiment of this invention.

FIG. 2 is a plan view of a containment tissue according to one embodiment of this invention.

FIG. 3 is a plan view of a containment tissue according to one embodiment of this invention.

FIG. 4 is a plan view of a containment tissue according to one embodiment of this invention.

FIG. 5 is a plan view of a containment tissue according to one embodiment of this invention.

FIG. 6 is a plan view of a containment tissue according to one embodiment of this invention.

FIG. 7 is a plan view of a containment tissue according to one embodiment of this invention.

FIG. 8 is a cross-sectional view of an absorbent article according to one embodiment of this invention.

FIG. 9 shows a process of printing a containment tissue according to one embodiment of this invention.

FIG. 10 shows a process of printing a containment tissue according to one embodiment of this invention.

FIG. 11 is a plan view of a Rewet apparatus used in the testing of this invention.

FIG. 12 is a graph of % Rewet of samples used to describe this invention versus pressure.

FIG. 13 is a graph of % Rewet of samples used to describe this invention versus pressure.

FIG. 14 is a graph of % Dryness Improvement of samples used to describe this invention versus pressure.

FIG. 15 is a graph of ISO Brightness % of samples used to describe this invention.

FIG. 16 is a graph of Color “L” factor of samples used to describe this invention.

FIG. 17 is a graph of ISO Brightness % of samples used to describe this invention used in a multilayer system.

FIG. 18 is a graph of Color “L” factor of samples used to describe this invention used in a multilayer system.

FIG. 19 is a graph of the DDA vacuum values of samples used to describe this invention.

FIG. 20 shows a plan view of a flexographic printing plate.

FIG. 21 shows a plan view of a flexographic printing plate.

FIG. 22 shows a plan view of a flexographic printing plate.

### DETAILED DESCRIPTION OF THE PRESENTLY PREFERRED EMBODIMENTS

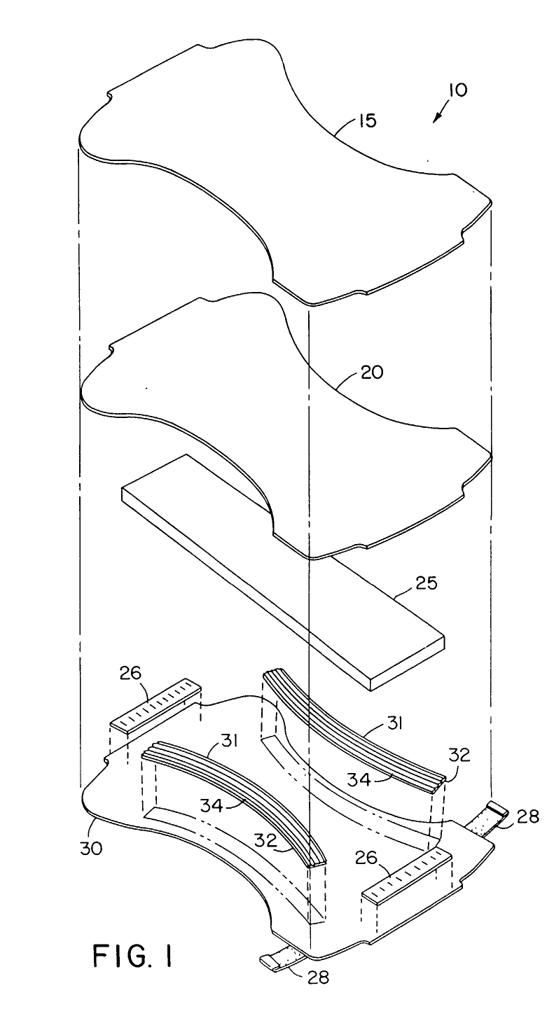

FIG. 1 illustrates an exploded perspective view of a disposable diaper. Referring to FIG. 1, disposable diaper 10 includes outer cover 30, body-side liner 15, containment tissue 20 adjacent to body-side liner 15, and absorbent core 25 located between containment tissue 20 and outer cover 30. Body-side liner 15 and outer cover 30 are constructed of conventional non-absorbent materials. By “non-absorbent” it is meant that the body-side liner 15 and outer cover 30 have an absorptive capacity not exceeding 5 grams of 0.9% aqueous sodium chloride solution per gram of material. INDA Standard Test Method IST 10.1 (95), “Standard Test Method for Absorbency Time, Absorbency Capacity, and Wicking Time,” published by INDA, Association of the Nonwoven Fabrics Industry, Cary, N.C., provides the basis for a suitable test method to measure absorbency. This test procedure is incorporated by reference.

Both containment tissue 20 and body-side liner 15 are constructed to be liquid pervious. These layers function to allow liquid from the wearer to contact the absorbent material or superabsorbent material present in diaper 10. Containment tissue 20 can embody any of the embodiments of this invention as disclosed herein or any equivalents. Body-side liner 15 can be made from materials including porous woven materials, porous nonwoven materials, films with apertures, open-celled foams, and batting.

Absorbent core 25 typically includes absorbent materials including natural fiber, such as wood pulp fibers, and/or nonwoven fibers or webs. “Nonwoven” and “nonwoven web” refer to materials and webs of material which are formed without the aid of a textile weaving or knitting process. Absorbent core 25 typically includes a matrix of hydrophilic fibers, such as a web of cellulosic fluff, mixed with particles of a superabsorbent material. Absorbent core 25 can also include absorbent foams generally known in the art including foams made by polymerizing high internal phase emulsions (HIPE's).

Outer cover material 30 should be breathable to water vapor but may be impermeable to vapor depending on the product. Outer cover 30 desirably includes a material that is substantially liquid impermeable, and can be elastic, stretchable or nonstretchable. The outer cover 30 can be a single layer of liquid impermeable material, but desirably includes a multi-layered laminate structure in which at least one of the layers is liquid impermeable. For instance, the outer cover 30 can include a liquid permeable outer layer and a liquid impermeable inner layer that are suitably joined together thermally, ultrasonically, by a laminate adhesive, or by any other suitable methods known in the art. Suitable laminate adhesives, which can be applied continuously or intermittently as beads, a spray, parallel swirls, or the like, can be obtained from Findley Adhesives, Inc., of Wauwatosa, Wis., U.S.A., or from National Starch and Chemical Company, Bridgewater, N.J., U.S.A. This invention or its embodiments is also suitable to function in the capacity of the inner layer of a multilayer outer cover 30. The liquid permeable outer layer can be any suitable material and desirably one that provides a generally cloth-like texture and/or mating fastening component qualities. One example of such a material is a 20 gsm (grams per square meter) spunbond polypropylene nonwoven web. The outer layer may also be made of those materials of which liquid permeable bodyside liner 15 is made. While it is not a necessity for the outer layer to be liquid permeable, it is desired that it provides a relatively cloth-like texture to the wearer.

The inner layer of the outer cover 30 can be both liquid and vapor impermeable, or can be liquid impermeable and vapor permeable. The inner layer is desirably manufactured from a thin plastic film, although other flexible liquid impermeable materials may also be used. The inner layer, or the liquid impermeable outer cover 40 when a single layer, prevents waste material from wetting articles, such as bedsheets and clothing, as well as the wearer and care giver. A suitable liquid impermeable film for use as a liquid impermeable inner layer, or a single layer liquid impermeable outer cover 30, is a 0.2 millimeter polyethylene film commercially available from Huntsman Packaging of Newport News, Va., U.S.A. If the outer cover 30 is a single layer of material, it can be embossed and/or matte finished providing a more cloth-like appearance. As earlier mentioned, the liquid impermeable material can permit vapors to escape from the interior of the disposable absorbent article, while still preventing liquids from passing through the outer cover 30. A suitable “breathable” material is composed of a microporous polymer film or a nonwoven fabric that has been coated or otherwise treated to impart a desired level of liquid impermeability. A suitable microporous film is a PMP-1 film material commercially available from Mitsui Toatsu Chemicals, Inc., Tokyo, Japan, or an XKO-8044 polyolefin film commercially available from 3M Company, Minneapolis, Minn.

Generally outer cover 30 will have a moisture vapor transmission rate (MVTR) of at least about 300 grams/m²-24 hours, preferably at least about 1000 grams/m²-24 hours, more preferably at least about 3000 grams/m²-24 hours, measured using INDA Test Method IST-70.4-99.

Attached to outer cover 30 are waist elastic members 26, fastening tapes 28 and leg elastic members 31. The leg elastics 31 comprise a carrier sheet 32, which can be a polyolefin film, and individual elastic strands 34. The waist elastic members 26 and the leg elastic members 31 can be formed of any suitable elastic material. As is well known to those skilled in the art, suitable elastic materials include sheets, strands or ribbons of natural rubber, synthetic rubber, or thermoplastic elastomeric polymers. The elastic materials can be stretched and adhered to a substrate, adhered to a gathered substrate, or adhered to a substrate and then elasticized or shrunk, for example with the application of heat; such that elastic constrictive forces are imparted to the substrate. In one particular embodiment, for example, the leg elastic members 31 include a plurality of dry-spun coalesced multifilament spandex elastomeric threads sold under the trade name LYCRA® and available from E. I. DuPont de Nemours and Company, Wilmington, Del., U.S.A.

The diaper of FIG. 1 is a general representation of one basic diaper embodiment. Various modifications can be made to the design and materials of diaper parts. The various layers of article 10 have dimensions which vary depending on the size and shape of the wearer.

Containment tissues of this invention are made of suitably natural fiber such as hardwood and softwood produced by various known processes including kraft processes, sulfite processes, or bleached chemi-mechanical processes (BCTMP). Two forms of containment tissues useful in this invention are an uncreped through air-dried (UCTAD) tissue material and a creped tissue material. Containment tissues can also include synthetic fibers, such as nonwoven fibers, added to the natural fiber base material.

Throughdrying provides a relatively noncompressive method of removing water from the web by passing hot air through the web until it is dry. More specifically, a wet-laid web is transferred from the forming fabric to a highly permeable throughdrying fabric and retained on the throughdrying fabric until dry. The resulting dried web is bulkier than a conventionally-dried uncreped sheet because fewer bonds are formed and because the web receives less compaction. Squeezing water from the wet web at a pressure roll is eliminated, although the use of a pressure roll elsewhere to subsequently transfer the web to a Yankee dryer for creping may still be used.

UCTAD (uncreped through air dried) tissue sheets are typically made by depositing a stream of an aqueous suspension of papermaking fibers onto a forming fabric where the web is partially dewatered to a consistency of about 10 dry weight percent. The forming fabric supports and carries the newly formed wet web while additional dewatering of the wet web can be carried out, such as by vacuum suction, while the wet web is supported by the forming fabric.

The wet web is transferred from the forming fabric to a transfer fabric that can be traveling at a slower speed than the forming fabric in order to impart increased machine direction stretch into the web. Minimal transfer pressure and dwell time is used to avoid compression of the wet web, preferably with the assistance of a vacuum shoe. Various designs of transfer fabrics can be used to impart the desired tissue properties.

The web is then transferred from the transfer fabric to the throughdrying fabric with the aid of a vacuum transfer roll or a vacuum transfer shoe. The throughdrying fabric can be traveling at about the same speed or a different speed relative to the transfer fabric. If desired, the throughdrying fabric can be run at a slower speed to further enhance machine direction stretch. Transfer is preferably carried out with vacuum assistance to ensure molding and deformation of the sheet to conform to the throughdrying fabric, thus yielding the desired thickness, stretch, air and liquid permeability, and texture. The design of the throughdrying fabrics can greatly affect the tissue properties. While supported by the throughdrying fabric, the web is dried by a throughdryer and transferred to a carrier fabric. The dried basesheet is transported to a reel for final collection of the tissue. Further description of UCTAD and production methods can be found in U.S. Pat. No. 5,607,551 issued on Mar. 4, 1997 to Farrington, Jr. et al., and is herein incorporated by reference.

As used herein, the “through air dried (TAD) side” of the tissue sheet is the side of the sheet facing the throughdrying fabric during throughdrying and the “air side” of the sheet is the side of the sheet facing away from the throughdrying fabric during throughdrying.

The “creped” tissue of this invention is produced by a papermaking process commonly known as the “dry crepe,” “light dry crepe,” or “Yankee creped” process. A “creped” containment sheet can be made as follows. Fibers of many varieties are slurried and then treated with suitable papermaking chemicals (e/g., dry and wet strength, dyes or colorants) either with batch or inline methods. The paper furnish is then pumped from a machine chest and flows to a headbox and through a slice or nozzle at about 0.05 to 0.5% consistency. The slurry is then dewatered and consolidated into a wet web on a forming wire or fabric. The forming system can be a Fourdrinier, twin-wire, or Crescent former as well as any other paper-machine apparatus used to dewater stock slurries.

The wet web is transferred to a carrier felt by contacting the web onto an absorbent felt by means of a couch roll or stationary element called a pick-up cam. The felt transports the web to a “wet press” assembly that accomplishes a significant amount of dewatering of the web as well as facilitates the attachment of the wet web to the Yankee dryer. The press assembly comprises either a single or double press roll usually accompanied by a vacuum system to aid in dewatering. The web simultaneously is dewatered and attached to the Yankee dryer at the nip between the Yankee dryer and the press roll. A significant amount of web compaction occurs in this nip.

The final drying and creping is accomplished on the Yankee dryer, a large steam-heated rotating drying cylinder. A portion of the Yankee dryer may be enclosed in a heated enclosure (“hood”) where heated air is directed to impinge on the web and assist in moisture removal by exhausting saturated air. The web is removed from the drying surface using a doctor blade creating a “creped” tissue. Thickness, stretch, and air and liquid permeability of the resultant “creped” tissue can be greatly affected by the design and orientation of the doctor blade as well as the creping additives that are applied to the Yankee dryer. Creping additives (e.g., adhesives and releases) are typically applied to Yankee surface using a spray system prior to attachment of the wet web.

The containment sheet of this invention can be single-ply products or multi-ply products, such as two-ply, three-ply, four-ply or greater. One-ply products are advantageous because of their lower cost of manufacture, while multi-ply products are preferred for improved properties. For multi-ply products it is not necessary that all plies of the product be the same, provided at least one ply is in accordance with this invention.

The basis weight of the containment tissues useful in this invention can be from about 5 to about 100 grams per square meter (gsm), suitably from about 10 to about 50 gsm, and more suitably from about 15 to about 30 gsm. For a single-ply containment tissue, a basis weight of about 30 gsm is suitable. For a two-ply tissue, a basis weight of about 15 gsm per ply is suitable. For a three-ply tissue, a basis weight of about 10 gsm per ply is suitable.

A wide variety of cellulosic and synthetic fibers can be employed in the process of creating the sheets/structures of the present invention. Many fiber types may be used for the present invention including hardwood or softwoods, straw, flax, milkweed seed floss fibers, abaca, hemp, kenaf, bagasse, cotton, canes and reeds, rice and esparto, bamboo and the like. All known papermaking fibers may be used, including bleached and unbleached fibers, fibers of natural origin (including wood fiber and other cellulose fibers, cellulose derivatives, and chemically stiffened or crosslinked fibers), some component portion of synthetic fiber (synthetic papermaking fibers include certain forms of fibers made from polypropylene, acrylic, aramids, acetates, and the like), virgin and recovered or recycled fibers, hardwood and softwood, and fibers that have been mechanically pulped (e.g., groundwood), chemically pulped (including but not limited to the kraft and sulfite pulp processings), thermomechanically pulped, chemi-thermomechanically pulped, and the like. Suitable wood sources include softwood sources such as pines, spruces, and firs, and hardwood sources such as oaks, eucalyptuses, poplars, beeches, and aspens.

Mixtures of any subset of the above mentioned or related fiber classes may be used. The pulp fibers can be prepared in a multiplicity of ways known to be advantageous in the art. Useful methods of preparing fibers include mechanical kneading or dispersion or chemical cross-linking to impart curl and improved web properties such as increased air and liquid permeability, higher thickness, and lower density. Examples of such mechanical treatment methods are disclosed in U.S. Pat. Nos. 5,348,620 issued Sep, 20, 1994 and 5,501,768 issued Mar. 26, 1996, both to M. A. Hermans et al., and U.S. Pat. No. 5,656,132 issued Aug. 12, 1997 to Farrington, Jr. et al.

Containment tissues can be imparted with a number of materials commonly used in the paper industry to impart wet strength to paper and board that are applicable to this invention. These materials are known in the art as wet strength agents and are commercially available from a wide variety of sources. Permanent, as opposed to temporary, wet strength agents will provide a more or less long-term wet resilience to the structure. This type of structure would find application in products that would require long-term wet resilience such as in paper towels, facial tissue and containment sheets in many absorbent products. The permanent wet strength agents that are of utility in the present invention are typically water soluble, cationic oligomeric, or polymeric resins that are capable of either crosslinking with themselves or with the cellulose or other constituent of the wood fiber. One of the most widely-used materials for this purpose is the class of polymer known as polyamide-polyamine-epichlorohydrin (PAE) type resins. These materials are sold by Hercules, Inc., Wilmington, Del., as Kymene 557H. Related materials are marketed by Henkel Chemical Co., Charlotte, N.C. and Georgia-Pacific Resins, Inc., Atlanta, Ga. With respect to the classes and the types of wet strength resins listed, it should be understood that this listing is simply to provide examples and that this is neither meant to exclude other types of wet strength resins useful in containment tissues of this invention.

It is generally desired that the cellulosic fibers used herein be wettable. As used herein, the term “wettable” is meant to refer to a fiber or material which exhibits a water-in-air contact angle of less than 90°. Suitably, the cellulosic fibers useful in the present invention exhibit a water-in-air contact angle of about 10° to about 50° and more suitably of about 20° to about 30°. Suitably, a wettable fiber refers to a fiber which exhibits a water-in-air contact angle of less than 90°, at a temperature of about 0° C. to about 100° C., and suitably at ambient conditions, such as about 23° C.

Suitable cellulosic fibers are those which are naturally wettable and are given the term “hydrophilic”. However, naturally nonwettable or “hydrophobic” fibers can also be used. “Hydrophilic” describes fibers or the surfaces of fibers which are wetted by the aqueous liquids in contact with the fibers. The degree of wetting of the materials can, in turn, be described in terms of the contact angles and the surface tensions of the liquids and materials involved. Equipment and techniques suitable for measuring the wettability of particular fiber materials or blends of fiber materials can be provided by a Cahn SFA-222 Surface Force Analyzer System, or a substantially equivalent system. When measured with this system, fibers having contact angles less than 90° are designated “wettable” or hydrophilic, while fibers having contact angles greater than 90° are designated “nonwettable” or “hydrophobic”. It is possible to treat the fiber surfaces by an appropriate method to render them and the resultant sheet, more or less wettable. When surface treated fibers are employed, the surface treatment is desirably non-fugitive; that is, the surface treatment desirably does not wash off the surface of the fiber with the first liquid insult or contact. For the purposes of this application, a surface treatment on a generally nonwettable fiber will be considered to be non-fugitive when a majority of the fibers demonstrate a water-in-air contact angle of less than 90° for three consecutive contact angle measurements, with drying between each measurement. That is, the same fiber is subjected to three separate contact angle determinations and, if all three of the contact angle determinations indicate a contact angle of water-in-air of less than 90°, the surface treatment on the fiber is considered to be non-fugitive. If the surface treatment is fugitive, the surface treatment will tend to wash off of the fiber during the first contact angle measurement, thus exposing the nonwettable surface of the underlying fiber, and will demonstrate subsequent contact angle measurements greater than 90°. Beneficial wettability agents include polyalkylene glycols, such as polyethylene glycols. The wettability agent is used in an amount comprising beneficially less than about 5 weight percent, suitably less than about 3 weight percent, and more suitably less than about 2 weight percent, of the total weight of the fiber, material, or absorbent structure being treated.

Containment sheets are often used in absorbent articles such as diapers, training pants, swimwear, feminine care products, and medical absorbent articles to prevent migration of superabsorbent material from the absorbent core 25 through the body-side liner 15, thereby decreasing superabsorbent material contact with the skin. However, after wetting many containment tissues are known to lose as much as 85% of the original dry tensile strength. Dry and wet tensile strength can be measured by a test method described below. With the additional stresses from body movements and the swelling absorbent core failure of containment tissues is common. “% Wet/Dry” is a common method to express the relative wet strength in a tissue. “% Wet/Dry” is the wet tensile strength divided by the dry tensile strength, multiplied by 100. It has been discovered that treating containment sheets with hydrophobic agents according to this invention increases the % Wet/Dry as well as the absolute wet tensile strength of the containment sheet. “Hydrophobic agents” refer to any chemical that can be applied to a containment sheet to impart hydrophobic characteristics to the hydrophobic agent treated region of the containment sheet. Examples of hydrophobic agents useful in this invention include without limitation, sizing agents, latexes, binder materials, adhesives and combinations of these. In one embodiment of this invention, the % Wet/Dry tensile strength of a treated region is at least about 15%, more suitably at least about 30%.

Chemical treatment of containment tissue 20 using a hydrophobic agent according to one embodiment of this invention is used to increase wet tensile strength. The increase in wet strength of containment tissue 20 is accomplished by imparting hydrophobicity to predetermined treated regions of containment tissue 20. The resulting containment tissue has hydrophilic regions where fluids can penetrate the containment tissue and hydrophobic regions where fluids are unable to penetrate the containment tissue. Various benefits can be obtained by controlling the amount and placement of the hydrophobic agent on containment tissue 20 according to this invention.

Hydrophobic agents can be applied to containment tissue 20 in various patterns, geometries and concentrations. “Treated region” refers to an area, or areas, of a containment tissue to which a hydrophobic agent such as a sizing agent, adhesive, or other hydrophobic chemistry is applied. Treated regions are also referred to as “hydrophobic regions.” There can be one treated regions or many treated regions on a single containment tissue. Treated regions vary in number, shape, and size depending on the needs of the various absorbent articles. “Untreated regions” or “hydrophilic regions” are regions of a containment sheet not treated with a hydrophobic agent according to this invention.

In one embodiment of this invention the hydrophobic agent is a sizing agent applied to containment tissue 20. A sizing agent imparts hydrophobic properties to the containment tissue providing hydrophobicity. Sizing agents are commonly added to control the penetration and spreading of aqueous liquids into paper or other fibrous structures. As used herein, a “sizing agent” is any chemical that imparts water repellency to a web matrix or individual cellulosic fibers. Suitable sizing agents and application methods are well known in the paper making arts. Internal sizing agents, which are those applied to the fibers within the paper structure, provide a reduced rate of penetration by retarding the rate of flow through the interfiber capillaries. When sizing is accomplished, the contact angle at the fiber surface is 90° or greater. Internal sizing agents function through the use of low surface energy, hydrophobic chemicals which when attached to the surface of cellulose reduce the surface energy of the fiber surface.

Suitable sizing agents are cationic molecules that bind to the fibers of containment tissue 20 and have a long hydrophobic chemical tail. Various sizing agents may be used according to this invention. Sizing agents useful in this invention include alkyl ketene dimers. Commercial sizing agents useful in this invention include, without limitation, Hercon 72S, Hercon 70, PTD M-1322, and Precis-2000, all from Hercules, Inc., Wilmington, Del. Particularly suitable sizing agents are acid or alkaline sizes such as acid rosin, alkenyl ketene dimers, alkenyl succinic anhydride (ASA), and alkenol ketene dimmers. Exemplary commercially available sizing agents of this type are Hercon 79 and Precis 3000 from Hercules, Inc., Wilmington, Del.

Latex and “binder material” compounds can also be used as hydrophobic agents to impart wet-strength and hydrophobicity to containment tissue 20. Examples include emulsion polymers such as thermoplastic vinyl acetate, a C1-C8 alkyl ester of acrylic or methacrylic acid based adhesive, or a combination of vinyl acetate and the C1-C8 alkyl ester of acrylic or methacrylic acid. The emulsion polymerized thermoplastic adhesive will have a glass transition temperature of about −25° C. to 20° C., a solids content of about 45% to 60% by weight, more suitably from 52% to 57%, and a Brookfield viscosity (#4 spindle, 60 rpm at 20° C.) of from 5 to 1000 centipoises (cps). Preferred adhesives are vinyl acetate/ethylene based adhesives incorporating less than about 10% and preferably less than 5% by weight, of a polymerized third monomer. Representative examples of third monomers which may be incorporated into the polymer include adhesion promoting monomers such as unsaturated carboxylic acid including acrylic and methacrylic acid, crotonic acid, and epoxide containing monomers such as glycidyl acrylate, glycidyl methacrylate and so forth. Commercially available examples include Airflex 401, 405 and 410, available from Air Products and Chemicals Inc., Allentown, Pa. In addition, crosslinkable thermoset binder materials can be used to impart further wet strength. The thermoset vinyl acetate/ethylene binder materials, such as vinyl acetate/ethylene having from 1-3% N-methylolacrylamide such as Airflex 124, 108 or 192, available from Air Products and Chemicals Inc., or Elite 22 and Elite 33, available from National Starch & Chemicals are suitable adhesive binder materials.

The emulsion polymerized thermoplastic polymeric adhesive is applied to the containment sheet or barrier tissue in an amount of from about 0.1 to 20 grams per square meter (gsm). Preferably about 0.3 to 5 grams dry adhesive per square meter by a print method whereby a reticulated or grid pattern of adhesive is coated onto the barrier tissue.

Hydrophobic silicone compounds can also be used as hydrophobic agents to impart wet-strength and hydrophobicity to containment tissue 20. As used herein, a “silicone” is any silicone polymer or oligomer having a silicon backbone, including polysiloxanes. The silicone can be unsubstituted polydimethylsiloxane or it can be a polysiloxane having substituted functional groups such as amino-, epoxy-, silanol-, or quaternary nitrogen.

Hydrophobic agents of this invention can be applied to a containment tissue through any method known in the art such as, without limitation, spraying or printing. Preferably the application process will allow application in a pattern of any shape or concentration. Printing methods such as gravure printing or flexographic printing allow the sizing agent to be easily applied in desired amounts and desired patterns. Other printing processes include using an inkjet printer to apply a sizing agent. The amount of hydrophobic agent added to the containment sheet can depend on product requirements and the size and basis weight of the containment sheet.

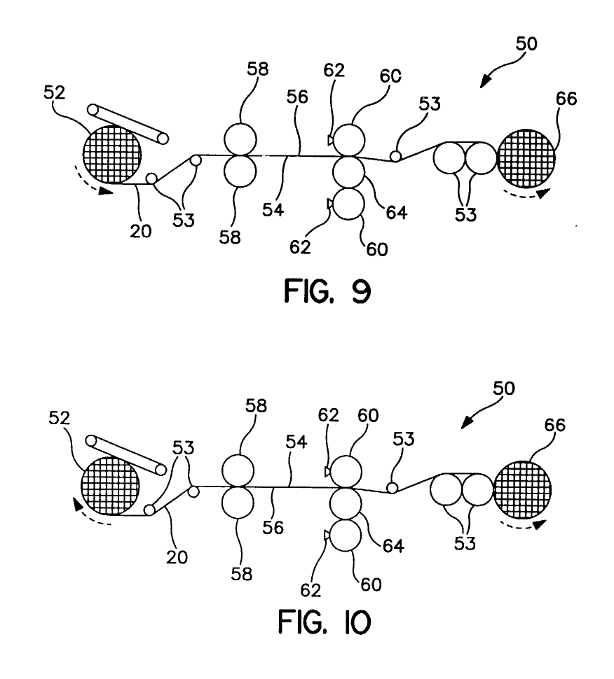

In one embodiment of this invention the printing of the hydrophobic agent is performed by a gravure printing process. Gravure printing processes are taught in U.S. Pat. No. 5,209,953, issued May 11, 1993 to Grupe et al., herein incorporated by reference. FIG. 9 shows a pilot converting line 50 configured to do gravure printing on containment tissue according to one embodiment of this invention. Pilot converting line 50 begins at parent roll 52 which is an UCTAD or creped parent roll of containment tissue 20. Parent roll 52 can rotate clockwise, as shown in FIG. 10, or counterclockwise, as shown in FIG. 9. UCTAD containment tissue 20 has two sides, through-air-dried (TAD) side 54 and air-side 56. In FIG. 9, if printing is from the bottom gravure printing roll 60 and intermediate roll 64, the set-up of parent roll 52 and the counterclockwise rotation allows printing on the TAD-side. In FIG. 10, the set-up of parent roll 52 and the counterclockwise rotation allows printing on the air-side.

After leaving parent roll 52, containment tissue travels over rollers 53 which provide direction and tension to the sheet of containment tissue 20. Optional calender rolls 58 are used to smooth the surface of containment tissue 20 to improve the uniformity of ink transfer to containment tissue 20. Containment tissue 20 can travel between calender rolls 58, as shown in FIGS. 9 and 10, or travel over a sequence of at least one calender roll 58.

Gravure printing of containment tissue can be accomplished by direct printing, indirect printing, or a combination of direct and indirect printing. Direct printing is shown in FIGS. 9 and 10 by the top gravure printing roll 60. Gravure printing roll 60 is an etched roll containing a pattern of depressions called “cells.”

The depressions on gravure printing roll 60 holds the chemical treatment, from applicator chamber 62, to be printed on containment tissue 20. During direct printing, gravure roll 60 directly contacts containment tissue 20 and the chemical treatment is transferred to containment tissue 20. Indirect printing occurs when the bottom gravure printing roll 60 transfers the chemical treatment to rubber intermediate roll 64 which then transfers the chemical treatment to containment tissue 20. After printing, containment tissue 20 contacts more rollers 53 before finishing on collection roll 66.

Gravure printing can be done to only one side or both sides of containment tissue 20, by either direct or indirect gravure printing. FIGS. 9 and 10 show a method of printing on both sides of containment tissue 20, with one side done by direct gravure printing and the other side done by indirect gravure printing. Alternatively, both sides could be printed by two direct gravure printings or two indirect gravure printings. By not filling one of applicator chamber 62 in pilot line 50, one can use only one gravure roll 60 to print on only one side of containment tissue 20.

Indirect gravure printing is generally more efficient than direct gravure printing in that the smooth rubber surface of intermediate roll 64 creates a better contact with the fibrous surface of containment tissue 20. Direct gravure printing typically achieves much lower transfer efficiency than the indirect gravure printing due to the non-uniform interface between gravure roll 60 and containment tissue 20. Also the transfer efficiency of direct gravure printing is dependent on the traveling speed of containment tissue 20, and therefore the rotation speed of gravure roll 60. This is due to the capillary effect of containment tissue 20 and the amount of chemical in the depressions of gravure roll 60, thus a slow traveling speed will likely result in a higher amount of chemical transferred to containment tissue 20. As there are no depressions in intermediate roll 64, the speed of containment tissue 20 is not as great a factor.

FIG. 9 also represents the configuration for a flexographic printing process. Flexographic printing is different from gravure printing by the type of roll in the intermediate roll 64 position and the source of the printed pattern. In the gravure process, intermediate roll 64 is a solid rubber surface that accepts the full pattern of the engraved or etched gravure roll 60 (lower). The flexographic configuration uses a magnetic roll substituted for the rubber intermediate roll 64. A rubber plate of the desired relief (herein called “pattern”) is attached to the magnetic roll 64. The key element of flexographic printing is the use of resilient relief image. plates that are placed on the magnetic roll 64. In the flexographic process, only the raised elements of the relief receive the chemical from roll 60. The chemicals are then deposited on containment tissue 20 in the pattern of the plate.

The amount of chemical printed by a gravure print roll is related to the cell volume of the depressions of the gravure roll per unit area. Gravure printing rolls are designated by the volume of print that can be transferred to containment tissue 20. This volume is typically expressed as cubic billion microns per square inch (cbm/in²) of gravure roll surface. As indirect printing is almost twice as efficient at transferring fluid to containment tissue 20 as direct printing, if both methods are used to print both sides of containment tissue 20, than the indirect printing gravure roll should be half the size of the roll used for the direct gravure printing.

The amount of sizing agent or equivalent compound printed onto containment tissue 20 is measured as the percent add-on. The “percent add-on” is the weight of the solid content of the sizing agent added to a containment tissue, or section of containment tissue, over the weight of the containment tissue, or section of containment tissue. Containment tissues of this invention have a sizing agent percent add-on of at least about 0.1%, more desirably a percent add-on of at least about 0.25%, and most desirably a percent add-on of at least about 0.5%.

Before, and during printing the hydrophobic agent is in the form of an emulsion. After printing the emulsion dries and the sizing agent solid is left on containment tissue 20.

Printing a sizing agent or equivalent compound onto containment tissue 20 decreases the thickness, or caliper, of containment tissue 20 in the treated region 21. Untreated containment tissues generally have a thickness from about 0.1 to 1.0 millimeters. Thickness of containment tissue 20 can be determined using an Emveco gauge, manufactured by Emveco, Inc.

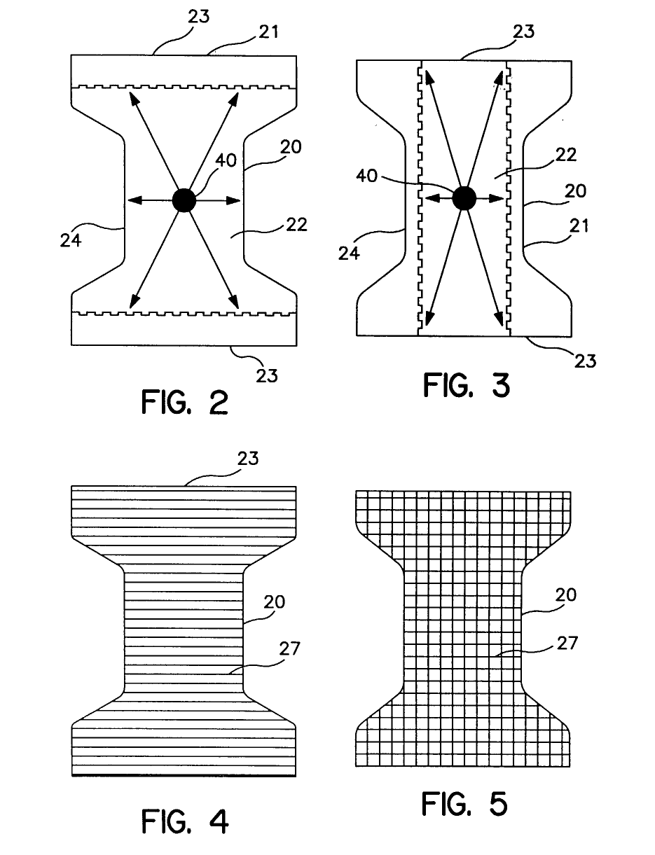

Containment tissues of this invention are useful in improving performance of absorbent articles. According to one embodiment of this invention the sizing agent is selectively applied to regions of containment tissue 20 resulting in less superabsorbent material contacting the user, decreased leakage, and improved skin health. FIG. 2 shows a containment tissue, useful in an absorbent article such as a diaper, according to one embodiment of this invention. A sizing agent is applied in treated region 21 of containment tissue 20. In FIG. 2 treated regions 21 are in an area of the waist regions 23 of the absorbent article. Untreated region 22 is between the two treated regions 21. FIG. 3 shows a containment tissue useful in an absorbent article such as a diaper, according to one embodiment of this invention. In FIG. 3 treated regions 21 extend from one waist region 23 to another waist region 23 along leg region 24. Untreated region 22 is between the two treated regions 21. Untreated regions of this invention can also be treated with surfactants to increase wettability.

The two embodiments of FIGS. 2 and 3 decrease fluid leakage through the waist regions 23 and leg regions 24, respectively, by treated regions 21. Without treated regions 21, fluid contacts containment tissue 20 and the fluid wicks throughout containment tissue 20, eventually reaching waist regions 23 and leg regions 24 where excess fluid can then leak out of the absorbent article. Application of a sizing agent in treated regions 21 stops wicking of any fluid at the boundary of treated regions 21. Fluid insult 40 represents fluid application in the target region of the absorbent article. The target region is located in the untreated regions 22 of FIGS. 2 and 3. The arrows extending from fluid insult 40 show the wicking of the fluid towards the edges of containment tissue 20. The wicking occurs substantially throughout untreated regions 22 and stops at the boundary of treated regions 21. Untreated region 22 allows fluid to penetrate containment tissue 20 and be absorbed by absorbent core 25. Sizing agents can be applied to containment tissue 20 in the patterns shown in FIGS. 4-7. FIG. 7 shows an application pattern that prevents wicking to the waist regions and the leg regions, thereby providing enhanced leakage control.

In one embodiment of this invention an adhesive compound, in addition to or instead of a sizing agent, is used as a hydrophobic agent to impart wet-strength to containment tissue 20. Containment tissue 20 can be reinforced with a curable or non-curing hydrophobic adhesive in a continuous, cross-liked network on the top or bottom of the tissue. Once applied, the adhesive may possess pressure-sensitive characteristics for securely attaching containment tissue 20 to other diaper components to prevent failure of the tissue. The adhesive can also be applied in regions to impart hydrophobic characteristics that will maintain dry-strength by preventing wetting. Adhesives can be attached to the tissue and subsequently cured to cause the adhesive to lose the adhesive properties yet still provide a strong, integral network. In one embodiment an adhesive will maintain adhesive qualities to be attached to other diaper components.

Adhesive can be applied to containment tissue 20 in various regions and/or geometries designed to accommodate key stress points, facilitate or restrict fluid absorption, maintain a specified open area, and/or minimize cost. Adhesives having stretch properties can be used to maintain flexibility of the absorbent article. Suitable adhesives useful in this invention include vinyl acetate and previously identified latexes and binder materials.

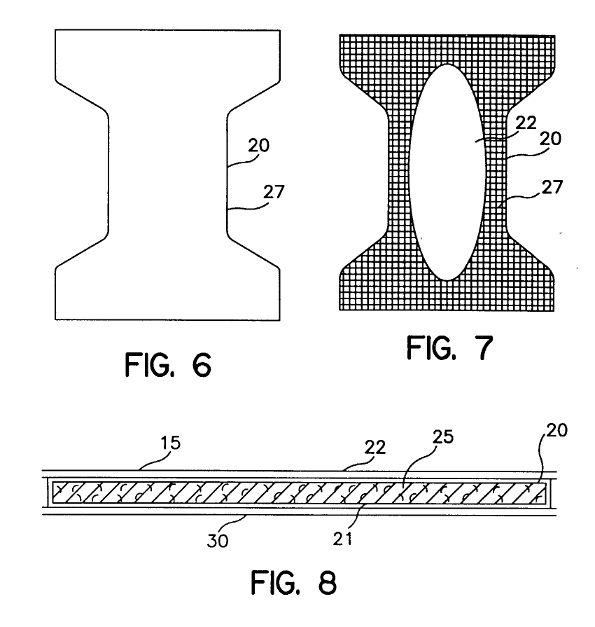

FIG. 4 shows containment tissue 20 with adhesive 27 applied in strips parallel to the waist regions 23. The application geometry of FIG. 4 has the benefit of increasing cross-direction strength and retaining the natural stretch of untreated containment tissue 20 in a direction perpendicular to the adhesive application. FIG. 5 shows one grid application geometry. The grid application geometry provides superior wet-strength and allows fluid penetration through the spaces between the adhesive 27 grid. FIG. 6 shows an embodiment of containment tissue 20 with adhesive 27 applied in a greater amount providing a less absorbent containment tissue 20. The embodiment of FIG. 6 has a lower overall fluid absorbency rate due to the decrease in untreated area through which fluids can flow. FIG. 7 shows a variation on the grid geometry. Untreated region 22 has no adhesive applied which allows maximize absorbency of fluid through containment tissue 20 to absorbent core 25. Adhesives can also be applied to containment tissue 20 as shown in FIGS. 2 and 3. Likewise, hydrophobic agents other than adhesives can be applied to containment sheets as shown in FIGS. 4-7

FIG. 8 shows one embodiment of this invention having containment tissue 20 wrapped around absorbent core 25. In this embodiment containment tissue is adjacent to a body-side liner 15 and an outer cover 30 of an absorbent article. Treated region 21 of containment tissue 20 covers the portion of absorbent core 25 towards the outer cover 30 of the absorbent article. Untreated region 22 of containment tissue 20 is between absorbent core 25 and the body-side liner 15. The boundaries of treated region 21 and untreated region 22 can be positioned to provide a suitable hydrophobic opening or openings on the side towards the user. The benefits of this embodiment include allowing fluid to flow to absorbent core through untreated region 22 of containment tissue 20 and treated region 21 of containment tissue 20 substantially impedes fluid flow from absorbent core 25 to the outer cover 30. Containment tissue 20 acts as a “bucket” to hold excess fluids in absorbent core 25. Wrapping absorbent core 25 in treated containment tissue 20 as shown in FIG. 8 is especially useful in feminine care absorbent articles.

Another use of containment tissues treated with a sizing agent or hydrophobic adhesive is as a visual indicator of wetness. The sizing agent or adhesive can be printed on a tissue in a shape that would be recognizable through the outer cover when the absorbent article is wetted. The printing can be done in shapes such as circles or ducks that would be beneficial in infant and toddler diapers. In some embodiments of this invention, the contrast between treated hydrophobic and wetted hydrophilic regions is used to achieve the visual indicator of the sheet and the contrast can typically be characterized by optical test methods well known in the art. This contrast is created by the brightness, color, and opacity differences of the non-wetted treated hydrophobic regions and wetted hydrophilic regions of the sheet. Any chemical treatment of this invention, including Hercon 70, Hercon 72S, and Precis 2000, are useful as visual indicators.

Hydrostatic head testing is used to determine the resistance of the treated areas of containment tissue 20 to liquid penetration under pressure. The hydrostatic head test is also used to evaluate the outer cover of diapers for leaks or pinholes under low hydrostatic pressure. The measured value is the hydrostatic head value reached when visible leakage is observed in three separate areas.

Hydrostatic head testing can be done using a Textest FX3000 Hydrostatic Head Tester, manufactured by Textest Ltd., Zurich, Switzerland. The pressure gradient is initially set at a low-end pressure gradient. The samples are positioned over a circular liquid reservoir and clamped into place along the outside edge. When the test is started the reservoir is increasingly pressurized and the liquid, usually water, pushes on the sample. When the first beads of liquid begin to appear on the surface of the sample the pressure is recorded as the hydrostatic head value.

Treated containment tissues according to this invention have a hydrostatic head value of at least about 5 millibars, desirably at least about 10 millibars, and more desirably at least about 16 millibars.

Containment tissues are made from porous materials to allow fluid to permeate the containment tissue and reach the absorbent core. “Porosity” of a containment tissue can be determined by a test that measures the air permeability of the containment tissue in terms of cubic feet of air per minute per square foot of tissue (cfm/min/ft²). The test can be performed using a Textest FX3300 air permeability tester manufactured by Textest Ltd., Zurich, Switzerland. The procedure used below was in accordance with TAPPI Method T 251, herein incorporated by reference. The instrument is set up and calibrated according to the manufacturer's manual. Single ply test specimens are cut large enough to completely extend beyond the clamping head. The test pressure is pre-selected for the test, which in the case of tissues and nonwovens is 125 Pa. A test head of 38 cm² was used.

In one embodiment of this invention the hydrophobic sizing agent is applied to regions of the containment sheet where ventilation is desirable, yet in a defined pattern to maintain the necessary fluid intake and distribution. Due to the porosity of the containment sheets, air exchange occurs easily through the containment sheet. However, the air permeability of the containment sheet is typically substantially impeded when the sheet is wetted. By applying a hydrophobic agent to regions on a containment sheet according to this invention, the air permeability is substantially or totally maintained due to the hydrophobic, non-wettable nature of the treated regions. The hydrophobic agent treated regions allow air exchange to occur while the surrounding hydrophilic areas generally do not. The treated regions patterns can be made contiguous or as discrete zones and therefore the breathable area can be varied depending on location within the absorbent article.

In one embodiment of this invention the containment tissue has at least two hydrophobic regions separated by at least one hydrophilic region. Suitably each hydrophobic region is at least about 1 mm² in size, more suitably each hydrophobic region is at least about 10 mm² in size, more suitably each hydrophobic region is at least about 75 mm² in size, and more suitably each hydrophobic region is at least about 150 mm² in size. In one embodiment of this invention the treated regions are of at least two sizes. Alternatively, in another embodiment of this invention, the containment tissue has at least two hydrophilic regions separated by at least one hydrophobic region. Suitably each hydrophilic region is at least about 1 mm² in size, more suitably each hydrophilic region is at least about 10 mm² in size, more suitably each hydrophilic region is at least about 75 mm² in size, and more suitably each hydrophilic region is at least about 150 mm² in size.

Containment tissues of this invention have a treated region porosity, or air permeability, value of greater than about 25 cfm/min/ft², more desirably an air permeability value of greater than about 60 cfm/min/ft², and preferably an air permeability value of greater than about 150 cfm/min/ft². After the absorbent article is wetted, these regions or “windows” will remain open despite the saturation in the neighboring hydrophilic areas. In the “wetted air permeability” test method described below, the air permeability of a control containment tissue is compared with containment tissues treated with patterned regions of hydrophobicity when both tissues are subjected to a wetted environment.

The ability of the treated hydrophobic regions of this invention to resist penetration of liquids in the z-direction can be characterized by many test methods well known in the art. One specific approach is to measure “rewet,” the amount of fluid that will penetrate the surface of the material when a load is applied. Absorbent systems can also be characterized by “flowback” tests, which may involve both the ability of the cover stock to resist penetration as well as the ability of the absorbent core to retain fluid.

The “Rewet Under Load” test procedure described below was designed to evaluate the ability of the hydrophobic regions to resist penetration of liquid from a partially saturated absorbent core over a range of pressures. Compression of the absorbent article during use is common and will exude free liquids from the absorbent material and bring the absorbent surface in closer proximity to the skin. Partial saturation is the typical state in the majority of the absorbent composites and products. This is especially true in non-target area as measured in fluid distribution profiles taken along the length of absorbent products. Full saturation is achieved in only small percentage of products and those occur generally at the end of the life cycle of the product.

The more fluid that penetrates through the surface and absorbs into the blotter paper, the higher the “rewet” value. Materials and absorbent systems with lower “rewet” result in less skin hydration, a major mechanism that causes damage to the stratum corneum, the outer protective skin layer. Therefore, removal of liquid and subsequent rewet prevention of the skin itself and of materials in proximity to the skin is vital to maintain skin containment protection.

As used herein, the “caliper” or thickness of a containment tissue is measured in accordance with TAPPI test method T402 “Standard Conditioning and Testing Atmosphere For Paper, Board, Pulp Handsheets and Related Products,” herein incorporated by reference, and is measured as one sheet using an EMVECO 200-A Microgage automated micrometer (EMVECO, Inc., Oregon). The micrometer has an anvil diameter of 2.22 inches (56.4 millimeters) and an anvil pressure of 132 grams per square inch (per 6.45 square centimeters) (2.0 kPa).

### EXAMPLES

Samples 1-8 were containment tissues used for Rewet testing and Optical Characterization testing described below. Samples 1-8 were made from various containment tissue and various sizing agents.

Samples 1 and 2 are made from UCTAD white containment tissue made with relatively low z-direction topography transfer and TAD fabrics containing 50% northern softwood kraft and 50% softwood BCTMP. Sample 1 was not treated with any hydrophobic agents and was hydrophilic. Sample 2 was uniformly treated with Hercon 70S sizing chemistry applied with indirect gravure printing using a 7.0 cbm pattern over 100% of the surface of the sheet. The Hercon 70S was at 12.5% solids, and had a percent add-on of approximately 1.5%.

Samples 3 and 4 were UCTAD white containment tissue made with relatively low topography transfer and TAD fabrics containing 33% northern softwood kraft and 67% softwood BCTMP. Sample 3 was not treated with any hydrophobic agents and was hydrophilic. Sample 4 was uniformly treated with Precis 2000 sizing chemistry applied with indirect gravure printing using a 7.0 cbm/in² pattern over 100% of the surface of the sheet. The Precis 2000 was at 26% solids, and had a percent add-on of approximately 1.9%.

Samples 5 and 6 were blue-dyed, wet-pressed, Yankee creped tissues containing approximately 50% northern softwood kraft and 50% northern hardwood kraft pulp. Sample 5 was not treated with any hydrophobic agents and was hydrophilic. Sample 6 was uniformly treated with Hercon 70S sizing chemistry applied with indirect gravure printing using a 4.0 cbm/in² pattern over 100% of the surface of the sheet. The Hercon 70 was at 12.5% solids, and had a percent add-on of approximately 0.7%.

Samples 7 and 8 were white wet-pressed, Yankee creped tissue containing approximately 50% northern softwood kraft and 50% northern hardwood kraft pulp. Sample 7 was not treated with any hydrophobic agents and was hydrophilic. Sample 8 was uniformly treated with Precis 2000 sizing chemistry applied with gravure printing using a 6.0 cbm/in² pattern over 100% of the surface of the sheet. The Precis 2000 was at 26% solids, and had a percent add-on of approximately 0.8%.

A summary of properties and attributes of Samples 1-8 is listed in Table 1, listing the values for basis weight, Emveco caliper, density, and air permeability.

TABLE 1

| | | | Basis | Emveco | | Air |
|---|---|---|---|---|---|---|
| Sam- | | Chemical | Weight | Caliper | Density | Permeability |
| ple | Color | Treatment | (gsm) | (mm) | (g/cm³) | (cfm/min/ft²) |
| 1 | White | None | 30.6 | 0.311 | 0.098 | 188.0 |
| 2 | White | Hercon 70S | 31.1 | 0.266 | 0.117 | 167.2 |
| 3 | White | None | 30.7 | 0.331 | 0.093 | 208.8 |
| 4 | White | Precis 2000 | 30.9 | 0.292 | 0.106 | 201.0 |
| 5 | White | None | 21.2 | 0.113 | 0.188 | 55.5 |
| 6 | White | Precis 2000 | 22.0 | 0.121 | 0.182 | 54.8 |
| 7 | Blue | None | 21.5 | 0.132 | 0.163 | 51.5 |
| 8 | Blue | Hercon 70S | 22.8 | 0.107 | 0.213 | 37.5 |

Rewet Under Load Testing

The Rewet Under Load (RUL) test procedure requires several preliminary preparations. Blotter paper (conditioned at 50% relative humidity) and a previously prepared absorbent composite material are both die-cut to 2⅜ inch diameter circles. The blotter and absorbent composite are die-cut to the same size to minimize horizontal wicking effects. In addition, an oversized blotter paper may absorb a disproportionate amount of liquid from the absorbent substrate causing a significant departure from the intended degree of saturation. Weigh and record the “dry” absorbent composite to the nearest 0.01-gram. Weigh and record each “dry” blotter to the nearest 0.01-gram.

The blotter paper used in this testing has a basis weight of approximately 354 grams per square meter (gsm), a 1-ply thickness of 0.735 mm, and a density of 0.48 g/cm³. Examples of commercially available blotter paper include VERIGOOD™ (available from Georgia Pacific, Atlanta, Ga.), or equivalent. The absorbent composite was produced on an air formed pilot line and composed of a relatively homogenous blend of 60% CR-1654 southern softwood pulp (from Alliance Forest Products) and 40% Favor 880 superabsorbent polymer (from Stockhausen Inc., Greensboro, N.C.) at a basis weight of 400 gsm and densified to 0.20 grams/cm³.

The saturation capacity of the absorbent composite was determined at 0.5 pounds per square inch (psi) and is used to calculate the amount of 0.9% by weight sodium chloride solution necessary to achieve the targeted % saturation. From the “dry” composite weight, calculate the amount of 0.9% by weight sodium chloride solution required to achieve the targeted % saturation for the absorbent composite. Add the required amount of 0.9% by weight sodium chloride solution to the absorbent composite and wait 15 minutes before testing. The saturation capacity of the pad used in the examples was 14.0 g/g. To determine how much liquid needs to be added to the pad to obtain 50% saturation, multiply the saturation capacity and the pad weight, and then multiply that number by 50%.

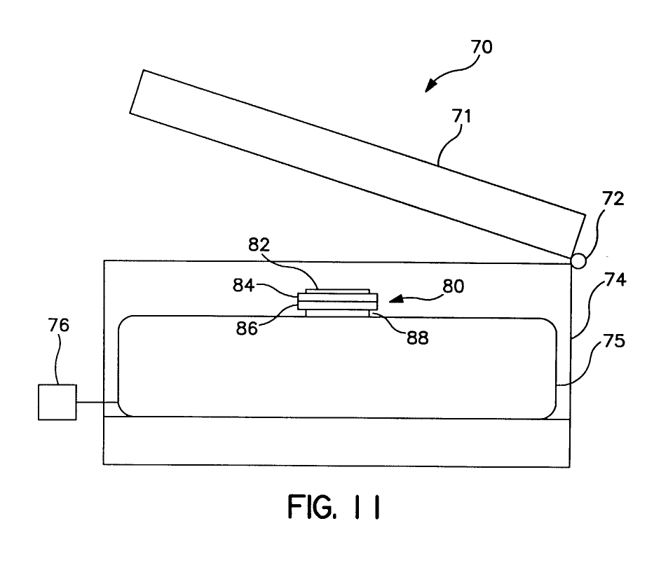

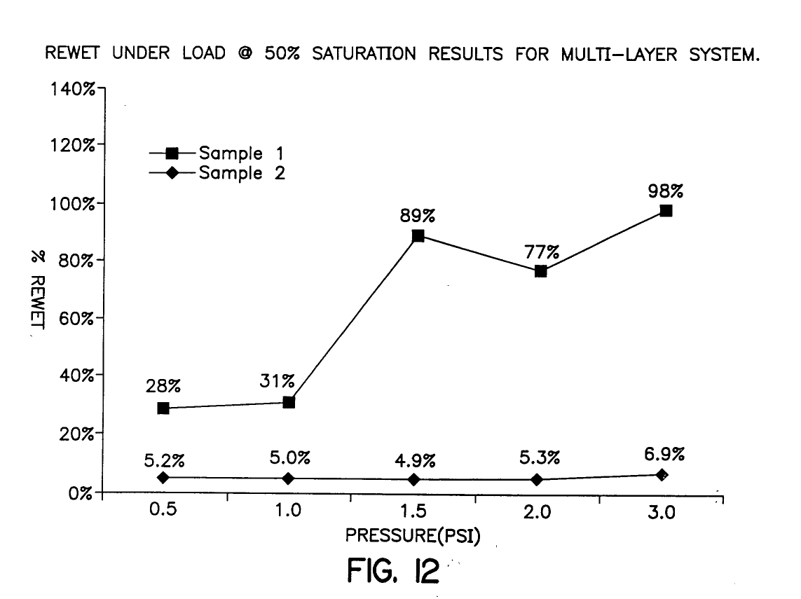

For RUL testing, each of Samples 1-8 were assembled into a two multilayer testing structures, testing structures 1 and 2. The first multilayer testing structure comprised the following components in the following order from top to bottom: blotter paper, sample containment sheet, and absorbent composite. The second multilayer testing structure comprised the following components in the following order from top to bottom: blotter, liner, sample containment sheet, and absorbent composite. In FIG. 11 the basic construction of second testing structure is designated by reference number 80. Second testing structure 80 comprises blotter paper 82, liner 84, sample containment sheet 86, and absorbent composite 88. First testing structure does not have a liner 84. Multilayer testing structure absorbent composite 88 and blotter paper 82 need to be vertically aligned and the containment sheet and liner should extend beyond the edges of the composite to prevent direct contact between the composite and blotter. A 10.16 cm×10.16 cm sample size is sufficient for the sample containment sheet 86 and liner 84. The construction of the specimen specified here can be adjusted to test the relationship of the invention with other various materials and multi-component structures that are used in absorbent articles.

For RUL testing, multilayer testing structure 80 is placed in the RUL testing device 70 as shown in FIG. 11. RUL testing device 70 comprises base 74, lid 71, hinge 72, air bladder 75, and pump 76. RUL testing device 70 applies the desired pressure in a controlled, uniform manner. Multilayer testing structure 80 is placed in the RUL testing device 70 so that the blotter paper 82 side is facing up and towards lid 71 and the absorbent composite 88 is in contact with air bladder 75. Five specimens were tested for each code unless noted. Testing was performed over a 0.5 to 3.0 psi pressure range using pump 76 that applied pressure by inflating air bladder 75 for a period of 5 minutes. The control systems allowed consistent ramp up to target pressure (15 to 20 seconds) and precise control of pressure (+/−0.1 psi) and test time (+/−0.01 seconds). This is desirable compared to other possible manual methods of using weights or adjustable jacks to apply the pressure.

The target air pressure (0.5, 1.0, 1.5, 2.0 or 3.0 psi) and time (5 minutes) are set on an operator panel. As the RUL testing device 70 is started, air bladder 75 inflates to the target pressure and depressurizes after the pre-set time interval of 5 minutes. Blotter paper 82 is removed immediately after deflation, weighed, and the “wet” value recorded to the nearest 0.01 gram. Calculate the amount of moisture absorbed by the blotter by subtracting the wet blotter weight from the dry blotter weight.

Rewet can be expressed in several different ways. First, the amount of moisture absorbed by the blotter paper can be reported in terms of grams. This can be used for the Rewet value if the blotter paper dry weights are reasonably close. The calculation is as follows:

Rewet(grams of liquid)=(Wet Blotter Wt.−Dry Blotter Wt.)

Second, “% Rewet” can be calculated by taking the amount of moisture absorbed by the blotter paper and dividing by the dry weight of the blotter paper. The calculation is as follows:

% Rewet=100×(Wet Blotter Wt.−Dry Blotter Wt.)/Dry Blotter Wt.

Third, the improvement in performance can be expressed by taking the difference of the % Rewet values of the specimens to be compared. For example, the % Rewet for the hydrophobic region of Specimen A minus the % Rewet value for the hydrophilic region of Specimen B is referred to as the “% Dryness Improvement,” thereby comparing Specimens A and B to each other. The calculation is as follows:

% Dryness Improvement(A, B)=% Rewet of A−% Rewet of B

The material and absorbent systems of this invention can be expressed in any of the three methods listed above as well as any other mathematical combination of measured values.

Samples 1-8 were tested by the above procedure to understand the differences in rewet behavior between hydrophobic and hydrophilic regions of a material when evaluated alone and as a multilayer system with other materials. Samples 1-4 possess relatively high air permeability and thickness, and low density compared to the creped tissues commonly used as containment sheets in commercial absorbent products such as PAMPERS Baby Dry® and HUGGIES UltraTrim® diapers. Samples 5 and 7 represent a class of creped tissues commonly used as containment sheets in commercial absorbent products such as PAMPERS Baby Dry® and HUGGIES UltraTrim® diapers. It will be demonstrated by the following RUL testing results that rewet behavior is dependent upon key physical attributes of the base material and surprisingly, not always in the obvious direction. In addition, a synergistic effect is obtained when the invention is placed adjacent to a liner material, with the liner material on the body or skin facing side of the absorbent article.

Each of Samples 1-8 were tested, as described above in multilayer testing structures, multiple times at pressures ranging from 0.5 psi to 3.0 psi. Each of Samples 1-8 was tested in a system without a spunbond liner (first multilayer testing structure) and with a spunbond liner (second multilayer testing structure) to compare the effects of the liner on rewet.

Table 2 summarizes the RUL test data at 50% initial saturation of the absorbent composite for Samples 1 and 2, and contains the subsequent calculations of Rewet (grams), % Rewet, and Dryness Improvement (%) as defined earlier.

TABLE 2

| | Pressure | | | Saline | Dry | Wet | | | Dryness |
|---|---|---|---|---|---|---|---|---|---|
| | Applied | Liner | Dry Pad | Added | Blotter | Blotter | Rewet | % | Improvement |
| Sample | (psi) | (Yes/No) | (g) | (g) | (g) | (g) | (g) | Rewet | (%) |
| 2 | 0.5 | Yes | 1.85 | 13.15 | 1.05 | 1.10 | 0.05 | 5.2% | 23.3% |
| 2 | 1.0 | Yes | 1.75 | 12.39 | 1.05 | 1.10 | 0.05 | 5.0% | 25.7% |
| 2 | 1.5 | Yes | 1.72 | 12.13 | 1.06 | 1.11 | 0.05 | 4.9% | 84.2% |
| 2 | 2.0 | Yes | 1.69 | 11.94 | 1.06 | 1.12 | 0.06 | 5.3% | 72.1% |
| 2 | 3.0 | Yes | 1.71 | 12.10 | 1.05 | 1.12 | 0.07 | 6.9% | 91.5% |
| 2 | 0.5 | No | 1.92 | 13.58 | 1.05 | 1.11 | 0.07 | 6.3% | 88.1% |
| 2 | 1.0 | No | 1.80 | 12.75 | 1.04 | 1.18 | 0.08 | 7.2% | 94.0% |
| 2 | 1.5 | No | 1.75 | 12.42 | 1.05 | 1.14 | 0.09 | 8.2% | 98.9% |
| 2 | 2.0 | No | 1.73 | 12.17 | 1.05 | 1.12 | 0.07 | 6.6% | 114.1% |
| 2 | 3.0 | No | 1.75 | 12.42 | 1.05 | 1.16 | 0.11 | 10.4% | 109.7% |
| 1 | 0.5 | Yes | 1.78 | 12.82 | 1.04 | 1.34 | 0.30 | 28.4% | |
| 1 | 1.0 | Yes | 1.76 | 12.61 | 1.05 | 1.37 | 0.32 | 30.6% | |
| 1 | 1.5 | Yes | 1.85 | 13.07 | 1.05 | 1.99 | 0.94 | 89.1% | |
| 1 | 2.0 | Yes | 1.76 | 12.40 | 1.07 | 1.89 | 0.83 | 77.3% | |
| 1 | 3.0 | Yes | 1.79 | 12.74 | 1.06 | 2.09 | 1.04 | 98.3% | |
| 1 | 0.5 | No | 1.66 | 11.78 | 1.05 | 2.03 | 0.99 | 94.4% | |
| 1 | 1.0 | No | 1.67 | 11.74 | 1.03 | 2.07 | 1.04 | 101.2% | |
| 1 | 1.5 | No | 1.79 | 12.66 | 1.05 | 2.17 | 1.12 | 107.1% | |
| 1 | 2.0 | No | 1.67 | 11.83 | 1.06 | 2.34 | 1.28 | 120.8% | |
| 1 | 3.0 | No | 1.81 | 12.86 | 1.05 | 2.32 | 1.27 | 120.2% | |
| Liner | 0.5 | Yes | 1.70 | 12.04 | 1.05 | 1.47 | 0.42 | 40.4% | |
| Liner | 1.0 | Yes | 1.73 | 12.50 | 1.04 | 1.80 | 0.76 | 73.2% | |
| Liner | 1.5 | Yes | 1.81 | 12.90 | 1.04 | 2.04 | 0.99 | 95.1% | |
| Liner | 2.0 | Yes | 1.76 | 12.34 | 1.06 | 2.10 | 1.03 | 97.1% | |
| Liner | 3.0 | Yes | 1.64 | 11.59 | 1.07 | 2.33 | 1.26 | 118.2% | |

FIG. 12 illustrates the difference between the % Rewet for the second multilayer structure (with liner) containing Samples 1 and 2. Across the entire pressure range of 0.5 to 3.0 psi, the multilayer structure containing Sample 2 essentially prevented any rewet of the blotter paper. For Sample 2, the average of 5.1% at the first four pressures is largely due to the water vapor absorbed by the blotter in the high humidity environment within the specimen while it is sandwiched in the test apparatus. However, the multilayer structure containing Sample 1 exhibits substantial rewetting at all pressures with an increasing trend in % Rewet as the applied pressure increases.

The unexpected result seen in the results of Table 2 is the ability of the hydrophobic Sample 2 to prevent rewet despite the high level of air permeability. The air permeability of Sample 2 (167 cfm/min/ft²) is much higher than any of the conventional creped tissues (from 37 to 56 cfm/min/ft²) shown in Table 1. The resistance to rewetting is robust over the range of very severe pressure loads that may occur during the use of absorbent articles.

The ability to remain hydrophobic and resistant to liquid penetration under load as demonstrated here will allow the inventions claimed to reliably provide other key product benefits (e.g., visual indicator, improved strength, less rewet, and breathability) discussed elsewhere.

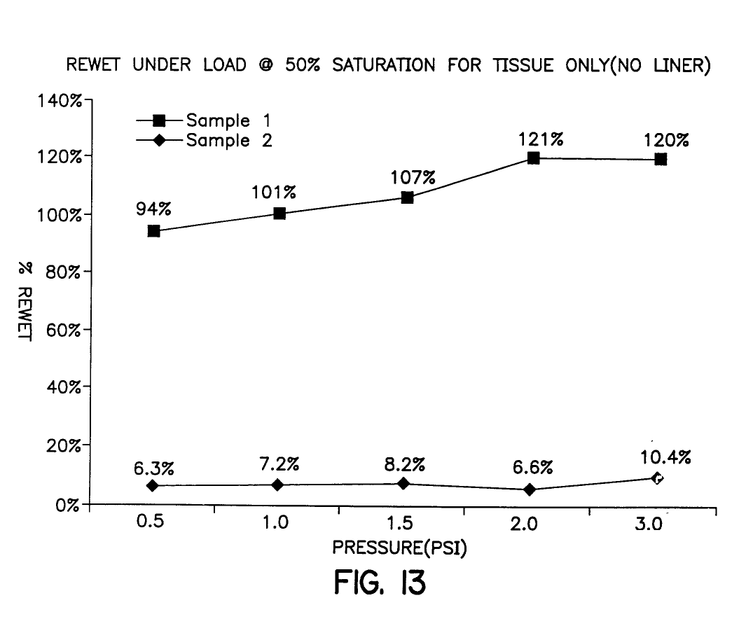

FIG. 13 compares the % Rewet performance of hydrophilic Sample 1 and hydrophobic Sample 2 in the first multilayer testing structure without a liner. Even without the liner, the hydrophobic Sample 2 tissue demonstrated excellent ability to minimize rewet of the blotter paper across the entire pressure range of 0.5 to 3.0 psi. Even at the highest pressure, Sample 2 only had a % Rewet of 10.4% (from the data for FIG. 13, approximately half of this value is attributed to absorption of water vapor). However, Sample 1 demonstrated significantly more % Rewet from 0.5 to 3.0 psi pressure. When in direct contact with the 50% saturated absorbent composite, Sample 1% Rewet ranges from 94% to 120% over that range. The unexpected result was the ability of Sample 2 to prevent rewet despite the high level of air permeability (167 cfm/min/ft²) and pressure and the absence of a liner on the blotter facing side.

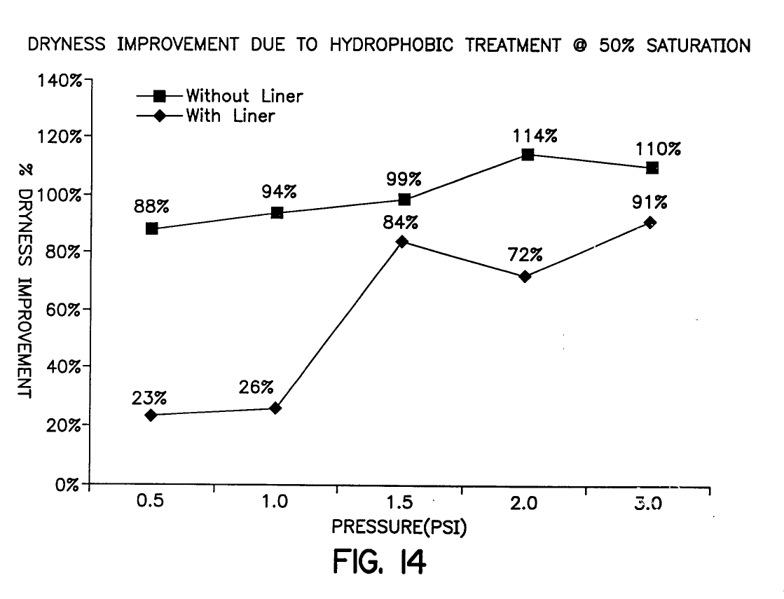

FIG. 14 demonstrates the Dryness Improvement % between Samples 1 and 2, achieved by the replacement of the hydrophilic Sample 1 with a hydrophobic Sample 2, for both individual sheets and multilayer structures over the 0.5 to 3.0 psi pressure range. In the absence of a liner, Sample 2 demonstrates a Dryness Improvement of about 88% to 114%. When Sample 2 is coupled with a liner to form the multilayer structure, a Dryness Improvement % of about 23% to 92% is achieved.

Table 3 summarizes the RUL data at 50% initial saturation of the absorbent composite for Samples 3 and 4, and contains the subsequent calculations of Rewet (grams), % Rewet, and Dryness Improvement (%) as defined earlier.

TABLE 3

| | Pressure | | Dry | Saline | | | | | |
|---|---|---|---|---|---|---|---|---|---|
| | applied | Liner | pad | added | Dry blotter | Wet blotter | REWET | % | % Dryness |
| Sample | (psi) | (Yes/No) | (g) | (g) | (g) | (g) | (g) | Rewet | Improvement |
| 4 | 0.5 | Yes | 1.73 | 12.28 | 1.01 | 1.08 | 0.06 | 6.3% | 13.4% |
| 4 | 1.0 | Yes | 1.70 | 12.06 | 1.02 | 1.08 | 0.07 | 6.5% | 24.3% |
| 4 | 1.5 | Yes | 1.73 | 12.27 | 1.03 | 1.08 | 0.05 | 5.0% | 65.7% |
| 4 | 2.0 | Yes | 1.74 | 12.33 | 1.03 | 1.08 | 0.06 | 5.4% | 53.5% |
| 4 | 3.0 | Yes | 1.79 | 12.57 | 1.01 | 1.07 | 0.06 | 6.1% | 76.4% |
| 4 | 0.5 | No | 1.71 | 12.12 | 1.03 | 1 09 | 0.06 | 6.0% | 74.2% |
| 4 | 1.0 | No | 1.70 | 12.00 | 1.03 | 1.10 | 0.07 | 6.6% | 97.5% |
| 4 | 1.5 | No | 1.75 | 12.37 | 1.03 | 1.10 | 0.06 | 6.2% | 83.0% |
| 4 | 2.0 | No | 1.63 | 11.53 | 1.01 | 1.12 | 0.11 | 10.7% | 90.6% |
| 4 | 3.0 | No | 1.75 | 12.42 | 0.99 | 1.57 | 0.58 | 58.5% | 48.2% |
| 3 | 0.5 | Yes | 1.74 | 12.29 | 1.02 | 1.23 | 0.20 | 19.7% | |
| 3 | 1.0 | Yes | 1.68 | 11.95 | 1.02 | 1.32 | 0.31 | 30.8% | |
| 3 | 1.5 | Yes | 1.80 | 12.58 | 1.04 | 1.77 | 0.73 | 70.8% | |
| 3 | 2.0 | Yes | 1.59 | 11.18 | 1.00 | 1.59 | 0.59 | 58.9% | |
| 3 | 3.0 | Yes | 1.75 | 12.33 | 0.99 | 1.81 | 0.82 | 82.6% | |
| 3 | 0.5 | No | 1.77 | 12.45 | 1.01 | 1.81 | 0.80 | 80.2% | |
| 3 | 1.0 | No | 1.85 | 12.98 | 1.01 | 2.05 | 1.05 | 104.1% | |
| 3 | 1.5 | No | 1.90 | 13.45 | 1.03 | 1.96 | 0.92 | 89.2% | |
| 3 | 2.0 | No | 1.69 | 11.91 | 1.01 | 2.02 | 1.02 | 101.3% | |
| 3 | 3.0 | No | 1.66 | 11.72 | 1.00 | 2.06 | 1.06 | 106.7% | |

The results from Samples 1-4 demonstrate several important features of this invention. First, a low density, the high air porosity hydrophilic tissue sheet of Sample 2 and 4 can have outstanding dryness results up to 2.0 and 3.0 psi loading for five minutes when treated with a sizing agent to become hydrophobic. Second, the multilayer structure comprising the Samples 2 and 4 containment sheets adjacent to additional components such as the liner can achieve dryness levels greater than the individual materials alone. Third, the multi-functional containment sheets resist rewetting over the 0.5 to 3.0 psi pressure range that represents severe conditions expected in typical absorbent products. This ability is necessary to achieve the other cited benefits claimed by the invention such as strength, dryness, breathability and visual indicator.

Overall, the % Rewet performance of the highly porous Samples 2 and 4 containment sheets was unexpectedly good in relation to the high air permeability. When treated to become hydrophobic, the three dimensional structure of UCTAD makes it difficult for liquids to pass through the tortuous pores. The hydrophobic nature of the surface, and tortuous and irregularly shaped pores of the UCTAD tissue structure, result in liquid bridging across the pore openings, unable to enter into and through the pores and through to the other side.

The sudden increase in % Rewet observed for Sample 4 at 3.0 psi suggests that a critical pore size distribution was exceeded for that pressure. When the pore size is finally increased sufficiently, the critical radius is exceeded and the liquid then overcomes resistance through numerous channels. This effectively happens as higher pore sizes are created with the higher level of softwood BCTMP (67% vs. 50%) and a lower density (0.106 vs. 0.117) for Sample 4 compared to Sample 2.

By incorporating the factors of thickness and pore size which effect penetration of a liquid through a porous hydrophobic web, a containment sheet of relatively large air permeability size can be designed to have an unexpectedly low % Rewet at specific pressures. Thickness will determine the effective pore length from one side of the sheet to the other, and the pore radius will define the necessary external pressure to overcome the bridging pressure of the liquid.

Table 4 summarizes the RUL data at 50% initial saturation of the absorbent composite for Samples 5 and 6, and contains the subsequent calculations of Rewet (grams), % Rewet, and Dryness Improvement (%) as defined earlier.

TABLE 4

| | Pressure | | | Saline | Dry | | | | |
|---|---|---|---|---|---|---|---|---|---|
| | Applied | Liner | | added | Blotter | Wet | | | % Dryness |
| Sample | (psi) | (Yes/No) | Dry Pad (g) | (g) | (g) | Blotter (g) | Rewet (g) | % REWET | Improvement |
| 6 | 0.5 | Yes | 1.70 | 11.96 | 1.03 | 1.09 | 0.06 | 6.0% | 13.9% |
| 6 | 1.0 | Yes | 1.74 | 12.35 | 1.02 | 1.09 | 0.07 | 6.7% | 24.5% |
| 6 | 1.5 | Yes | 1.77 | 12.61 | 1.01 | 1.08 | 0.07 | 6.9% | 36.5% |
| 6 | 2.0 | Yes | 1.70 | 12.03 | 1.01 | 1.08 | 0.07 | 6.9% | 72.2% |
| 6 | 3.0 | Yes | 1.73 | 12.19 | 1.00 | 1.09 | 0.09 | 9.2% | 84.2% |
| 6 | 0.5 | No | 1.71 | 12.01 | 1.02 | 1.10 | 0.07 | 7.0% | 75.8% |
| 6 | 1.0 | No | 1.78 | 12.49 | 1.06 | 1.15 | 0.09 | 8.9% | 80.3% |
| 6 | 1.5 | No | 1.82 | 12.79 | 1.02 | 1.11 | 0.09 | 8.8% | 80.4% |
| 6 | 2.0 | No | 1.66 | 11.72 | 1.02 | 1.34 | 0.32 | 31.4% | 88.1% |
| 6 | 3.0 | No | 1.72 | 12.15 | 1.01 | 1.76 | 0.74 | 73.5% | 42.0% |
| 5 | 0.5 | Yes | 1.76 | 12.47 | 1.01 | 1.21 | 0.20 | 20.0% | |
| 5 | 1.0 | Yes | 1.75 | 12.34 | 1 03 | 1.35 | 0.32 | 31.1% | |
| 5 | 1.5 | Yes | 1.72 | 12.15 | 1.03 | 1.47 | 0.45 | 43.5% | |
| 5 | 2.0 | Yes | 1.73 | 12.15 | 1.01 | 1.81 | 0.80 | 79.1% | |
| 5 | 3.0 | Yes | 1.71 | 12.03 | 1.01 | 1.95 | 0.94 | 93.4% | |
| 5 | 0.5 | No | 1.74 | 12.26 | 1.02 | 1.86 | 0.84 | 82.8% | |
| 5 | 1.0 | No | 1.75 | 12.36 | 1.04 | 1.96 | 0.92 | 89.1% | |
| 5 | 1.5 | No | 1.75 | 12.32 | 1.04 | 1.97 | 0.93 | 89.2% | |
| 5 | 2.0 | No | 1.69 | 11.99 | 1.04 | 2.29 | 1.24 | 119.4% | |
| 5 | 3.0 | No | 1.68 | 11.93 | 1.03 | 2.23 | 1.19 | 115.5% | |

The results of Sample 6 agree with the trends observed for Samples 2 and 4, that the addition of a hydrophobic agent decreases rewet in containment tissues.

Table 5 summarizes the RUL data at 50% initial saturation of the absorbent composite for Samples 7 and 8, and contains the subsequent calculations of Rewet(grams), % Rewet, and Dryness Improvement (%) as defined earlier.

TABLE 5

| | Pressure | | | | | Wet | | | Dryness |
|---|---|---|---|---|---|---|---|---|---|
| | Applied | Liner | Dry Pad | Saline Added | Dry Blotter | Blotter | | | Improvement |
| Sample | (psi) | (Yes/No) | (g) | (g) | (g) | (g) | Rewet (g) | % Rewet | (%) |
| 8 | 0.5 | Yes | 1.74 | 12.30 | 1.02 | 1.08 | 0.06 | 5.7% | 20.9% |
| 8 | 1.0 | Yes | 1.74 | 12.38 | 1.01 | 1.07 | 0.07 | 6.6% | 28.2% |
| 8 | 1.5 | Yes | 1.79 | 12.70 | 1.00 | 1.12 | 0.11 | 11.1% | 66.7% |
| 8 | 2.0 | Yes | 1.79 | 12.61 | 1.00 | 1.34 | 0.34 | 33.8% | 49.4% |
| 8 | 3.0 | Yes | 1.80 | 12.67 | 1.00 | 1.78 | 0.78 | 77.6% | 23.4% |
| 8 | 0.5 | No | 1.77 | 12.61 | 1.01 | 1.29 | 0.28 | 27.6% | 40.3% |
| 8 | 1.0 | No | 1.76 | 12.41 | 1.01 | 1.56 | 0.55 | 54.4% | 39.1% |
| 8 | 1.5 | No | 1.69 | 11.88 | 1.02 | 1.51 | 0.49 | 47.6% | 60.0% |
| 8 | 2.0 | No | 1.69 | 11.94 | 0.99 | 1.81 | 0.82 | 83.0% | 25.2% |
| 8 | 3.0 | No | 1.73 | 12.20 | 1.01 | 1.95 | 0.94 | 93.8% | 17.0% |
| 7 | 0.5 | Yes | 1.74 | 12.37 | 1.01 | 1.28 | 0.27 | 26.6% | |
| 7 | 1.0 | Yes | 1.71 | 12.12 | 1.00 | 1.35 | 0.35 | 34.7% | |
| 7 | 1.5 | Yes | 1.80 | 12.75 | 1.02 | 1.81 | 0.79 | 77.8% | |
| 7 | 2.0 | Yes | 1.72 | 12.19 | 1.00 | 1.83 | 0.83 | 83.2% | |
| 7 | 3.0 | Yes | 1.73 | 12.19 | 1.00 | 2.00 | 1.01 | 101.0% | |
| 7 | 0.5 | No | 1.76 | 12.41 | 1.00 | 1.68 | 0.68 | 67.9% | |
| 7 | 1.0 | No | 1.75 | 12.34 | 1.00 | 1.94 | 0.94 | 93.5% | |
| 7 | 1.5 | No | 1.76 | 12.44 | 1.02 | 2.11 | 1.09 | 107.6% | |
| 7 | 2.0 | No | 1.72 | 12.12 | 1.01 | 2.11 | 1.09 | 108.2% | |
| 7 | 3.0 | No | 1.74 | 12.27 | 1.01 | 2.12 | 1.12 | 110.8% | |

Across the entire pressure range of 0.5 to 3.0 psi, the multi-layer system containing Sample 8 essentially prevented any rewet of the blotter paper. For Sample 8, the average % Rewet of 7.1% is largely due to the water vapor absorbed by the blotter in the high humidity environment within the specimen while it is sandwiched in the test apparatus. The multilayer structure containing Sample 7 exhibits substantial rewetting at all pressures with an increasing trend in % Rewet with applied pressure.

Again, the unexpected result was the ability of the hydrophobic Sample 8 to event rewet over the pressure range tested despite its air permeability of 37.5 cfm/min/ft² and relatively low 1-ply thickness of 0.107 mm as shown in Table 1. The resistance to rewetting is robust up to very severe loadings that may occur during the use of absorbent articles. This agrees with the trends observed for Samples 2, 4, and 6. The ability to remain hydrophobic and resistant to liquid penetration under load as demonstrated here will allow the inventions claimed to reliably provide other key product benefits (e.g., visual indicator, improved strength, less rewet, more breathability) cited elsewhere.

Brightness and Color Testing

In one method of optical testing of the visual indicator characteristics of some embodiments of this invention, each region, treated and non-treated, is characterized individually either as a homogenous material or as part of a multilayer or multicomponent system. The brightness and color components were measured using a Technibrite Micro TB-1C meter (available from Technidyne Corporation, New Albany, Ind., USA). Calibration and operating procedures were followed from the Instruction Manual.

“Brightness” herein is defined as “% ISO Brightness.” “Color” herein is defined as “L”, “a”, and “b” factors, and expressed as a percentage. “L” represents the percent diffuse reflectance (brightness) which ranges from 0 (black) to 100 (white). The “a” value is a measure of the redness (+a) and greenness (−a). The “b” value is a measure of yellowness (+b) and blueness (−b). For both the “a” and “b” values, the greater the departure from 0, the more intense the color. The specific parameters provided here are reported by selecting “0—Brightness” and “4—L, a, b” on the Technibrite instrument. It is recognized that many other related optical parameters exist in color theory and these measurements could be generated to characterize the nature of the invention. Alternative measurements are, but not limited to, “R(x), R(y), R(z)”, “X, Y, Z”, and “L*, a*, b*”. Absorption and scattering coefficients as well as opacity could also be used characterize the difference in visual properties achieved as a result of the invention.

All test specimens were constructed with multiple plies to provide “infinite thickness” to avoid penetration of the light source through the bottom of the samples. In color theory, the value measured when all light is either absorbed or scattered by the substrate is generally called “reflectivity” or R(∞). Preliminary testing indicated that 20 plies provided infinite thickness for the dry hydrophobic samples and 30 plies provided infinite thickness for the wetted hydrophilic samples. All subsequent testing used 20 or 30 plies depending upon the nature of the samples. Each sample for optical testing was prepared by cutting a 3-inch by 3-inch square and then stacking up the required number of plies.

The first series of test specimens were prepared in pairs, with one sample representing a hydrophobic sheet treated in the method of the invention and a hydrophilic sheet. It was desirable to originate both specimens from the same base material to avoid variation in composition. A brightness and color measurement was then taken from the dry 20-ply hydrophobic specimen. Next, the hydrophilic specimen was wetted with 0.9% by weight sodium chloride solution prior to placement in the Technibrite holder in the amount to give greater than 50% and less than 100% saturation of the hydrophilic portion of the specimen. The sodium chloride solution was allowed to uniformly spread through the specimen. The amount of sodium chloride solution added was less than required for total saturation of the specimen in order to avoid pooling of the liquid. A brightness and color measurement was then taken from the saturated hydrophilic specimen. Three measurements were taken for each code and the average and standard deviation reported. The moisture content of each wetted specimen was also measured after completion of the test to verify the degree of saturation.

The measure of contrast, herein defined as “Visual Contrast”, was then calculated by taking the brightness or color value from the hydrophobic sample and subtracting the value of the corresponding hydrophilic sample. The optical parameters calculated in this manner are referred to as “visual contrast” in general and “Brightness Visual Contrast,” “L Factor Visual Contrast,” “‘a’ Factor Visual Contrast,” and “‘b’ Factor Visual Contrast.” The following are representative calculations for these contrast measurements.

ISO Brightness Visual Contrast=ISO Brightness (Hydrophobic)−ISO Brightness (Hydrophilic)

L Factor Visual Contrast=L(Hydrophobic)−L(Hydrophilic)

Samples 1-8 were tested to illustrate the “Visual Contrast” values that can be achieved by creating regions of hydrophobic and hydrophilic areas. The optical test method used was as described above as well as the computation for “Visual Contrast.”

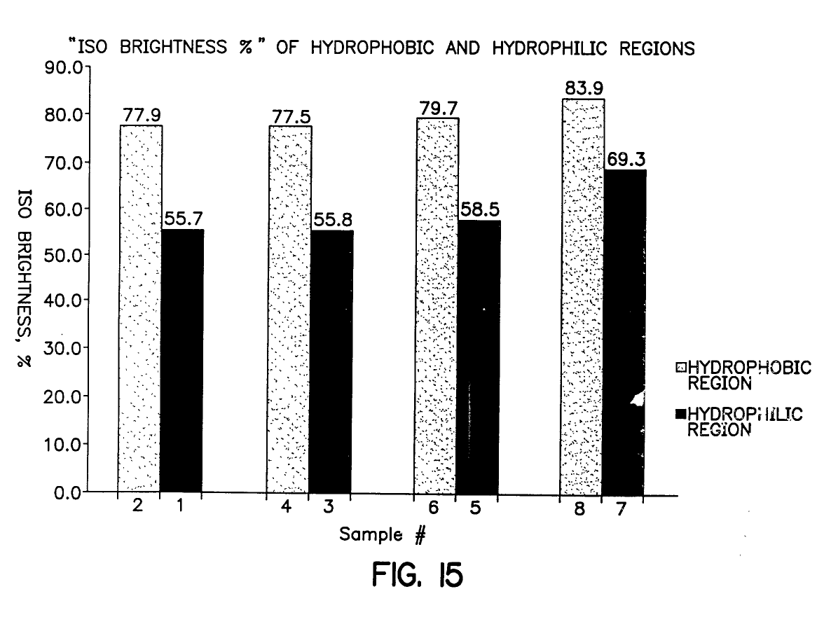

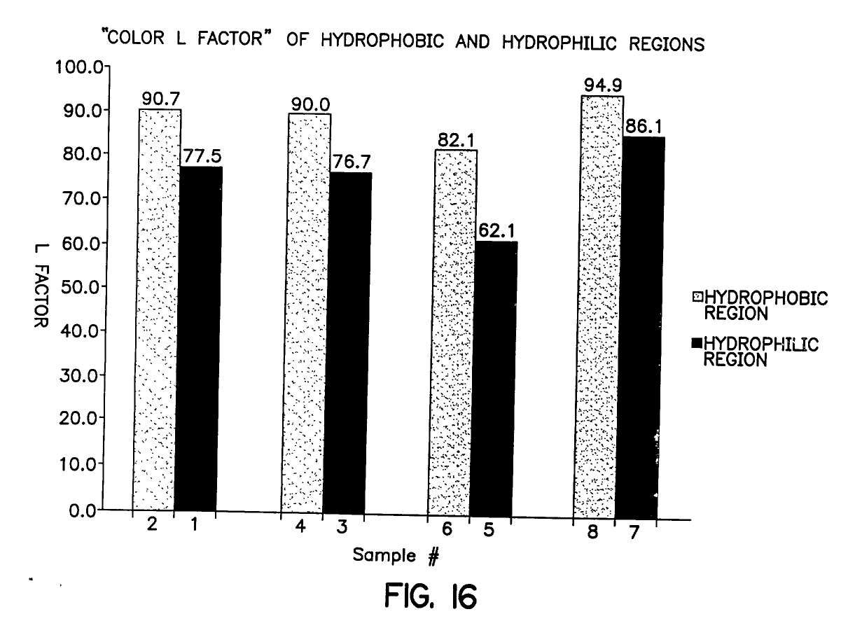

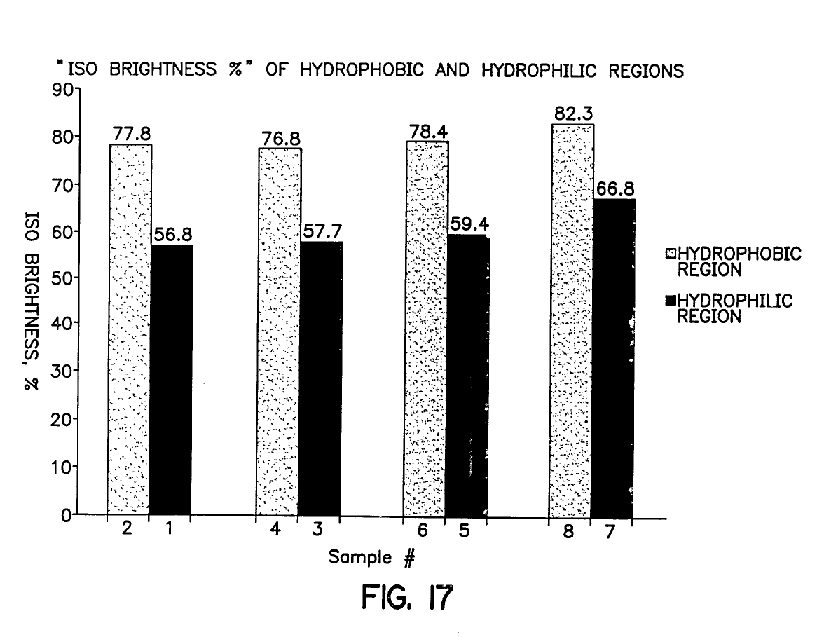

Table 6 summarizes the ISO Brightness and the color (L, A and B factors) averages and standard deviation (STD) measured for each of the samples. FIGS. 15 and 16 are graphs comparing the brightness and color results of the hydrophobic region and hydrophilic region for each sample in Table 6. The difference in height between each pair of sample bars (i.e. Samples 1 and 2) is the “Visual Contrast” for that tissue.

TABLE 6

| | | AVG or | ISO | | | |
|---|---|---|---|---|---|---|
| Sample | # of Plies | STD | Bright (%) | L | a | b |
| 1 | 30 | Average | 55.70 | 77.53 | −0.48 | 5.08 |
| | | STD | 0.62 | 0.68 | 0.08 | 0.41 |
| 2 | 20 | Average | 77.87 | 90.70 | 0.24 | 4.12 |
| | | STD | 0.29 | 0.35 | 0.22 | 0.12 |
| 3 | 30 | Average | 55.77 | 76.70 | −0.10 | 4.02 |
| | | STD | 1.14 | 0.95 | 0.11 | 0.34 |
| 4 | 20 | Average | 77.53 | 90.03 | 0.67 | 3.41 |
| | | STD | 0.25 | 0.06 | 0.03 | 0.17 |
| 5 | 30 | Average | 58.50 | 62.10 | −7.93 | −22.00 |
| | | STD | 0.26 | 0.17 | 0.12 | 0.35 |
| 6 | 20 | Average | 79.73 | 82.07 | −5.17 | −10.37 |
| | | STD | 0.15 | 0.06 | 0.06 | 0.21 |
| 7 | 30 | Average | 69.27 | 86.13 | −0.79 | 4.29 |
| | | STD | 0.12 | 0.38 | 0.10 | 0.54 |
| 8 | 20 | Average | 83.87 | 94.93 | −0.54 | 4.71 |
| | | STD | 0.29 | 0.06 | 0.04 | 0.20 |

Table 7 lists the computed Visual Contrast between the hydrophobic and hydrophilic regions for ISO Brightness and L, a, and b values. The change in brightness created when the hydrophilic region is wetted resulted in a significant visual contrast with the corresponding dry hydrophobic region for all tissue types.

TABLE 7

| Samples | ISO | | | |
|---|---|---|---|---|
| Compared | Bright | L | a | b |
| 1 and 2 | 22.17 | 13.17 | 0.72 | −0.96 |
| 3 and 4 | 21.77 | 13.33 | 0.77 | −0.61 |
| 5 and 6 | 21.23 | 19.97 | 2.77 | 11.63 |
| 7 and 8 | 14.60 | 8.80 | 0.24 | 0.42 |

Unexpectedly, comparison of the hydrophobic and the wetted hydrophilic the blue tissue (Samples 5 and 6) demonstrated the largest change in all three of the color values. The visual contrasts of the “L”, “a”, and “b” factors were 19.97, 2.77 and 11.63, respectively. The dominating color, in this case blue, is darkened leading to a dramatic change in “b” factor. This change in the dominating color factor can be used advantageously to create subtle or striking visual cue effects based on the original dry color of the base material.

A second series of Samples 1-8 including a single ply spunbond liner placed on the top of a multi-ply tissue stack was then tested using the Technibrite Micro TB-1C as described above. The underlying plies of each specimen consisted of the hydrophobic sample treated in the method of the invention or a hydrophilic sample. It was desirable to originate both samples from the same base material to avoid variation in composition. The hydrophobic samples tested comprised 20 plies and the corresponding hydrophilic samples comprised 30 plies. A brightness and color measurement was then taken from a dry hydrophobic specimen. Next, the hydrophilic specimen was wetted with 0.9% by weight sodium chloride solution prior to placement in the Technibrite holder in the amount to give greater than 50% and less than 100% saturation of the hydrophilic portion of the specimen. The sodium chloride solution was allowed to uniformly spread through the specimen. The amount of sodium chloride solution added was less than required for total saturation of the specimen in order to avoid pooling of the liquid. A brightness and color measurement was taken from the wetted hydrophilic specimen. Three measurements were taken for each sample and the average and standard deviation reported. The moisture content of each wetted specimen was also measured after completion of the test to verify the degree of saturation insuring that it was greater than 50% and less than 100%.

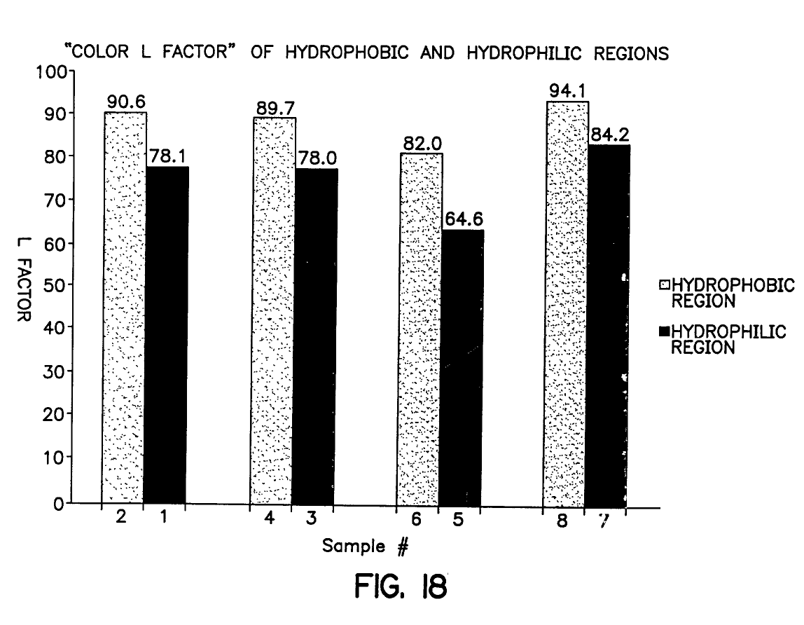

Table 8 lists the ISO Brightness and the color (L, a, and b factors) average and standard deviation measured for each multi-component sample. FIGS. 17 and 18 are graphs comparing the brightness and color results of the hydrophobic region and hydrophilic region for each multilayer sample of Table 8. The difference in height between each pair of bars is the “Visual Contrast” for that multi-component system.

TABLE 8

| | | AVG OR | ISO | | | |
|---|---|---|---|---|---|---|
| Sample | # of Plies | STD | Bright (%) | L | a | b |
| 1 | 30 | Average | 56.76 | 78.09 | −0.71 | 4.89 |
| | | STD | 1.09 | 0.74 | 0.09 | 0.08 |
| 2 | 20 | Average | 77.75 | 90.55 | 0.28 | 3.94 |
| | | STD | 0.15 | 0.04 | 0.04 | 0.02 |
| 3 | 30 | Average | 57.72 | 77.99 | −0.26 | 3.99 |
| | | STD | 0.27 | 0.17 | 0.03 | 0.08 |
| 4 | 20 | Average | 76.80 | 89.74 | 0.36 | 3.60 |
| | | STD | 0.21 | 0.04 | 0.01 | 0.11 |
| 5 | 30 | Average | 59.37 | 64.64 | −7.42 | −18.47 |
| | | STD | 1.02 | 1.18 | 0.20 | 0.87 |
| 6 | 20 | Average | 78.42 | 81.96 | −4.85 | −9.30 |
| | | STD | 0.07 | 0.02 | 0.03 | 0.06 |
| 7 | 30 | Average | 66.77 | 84.21 | −1.07 | 3.77 |
| | | STD | 0.68 | 0.25 | 0.03 | 0.31 |
| 8 | 20 | Average | 82.30 | 94.10 | −0.84 | 4.96 |
| | | STD | 0.14 | 0.01 | 0.05 | 0.10 |

Table 9 lists the computed Visual Contrast between the hydrophobic and hydrophilic regions for ISO Brightness and “L”, “a” and “b” factors. The change in brightness created when the hydrophilic region is wetted resulted in a significant visual contrast with the corresponding dry hydrophobic region for all tissue types. Table 9 also lists the Sample comparisons from Table 7 (tissue only) for comparison.

TABLE 9

| Samples | | | | | |
|---|---|---|---|---|---|
| Compared | Configuration | ISO Bright | L | a | b |
| 1 and 2 | Tissue Only | 22.17 | 13.17 | 0.72 | −0.96 |
| | Multi-layer | 20.99 | 12.46 | 0.99 | −0.95 |
| 3 and 4 | Tissue Only | 21.77 | 13.33 | 0.77 | −0.61 |
| | Multi-layer | 19.07 | 11.75 | 0.62 | −0.38 |
| 5 and 6 | Tissue Only | 21.23 | 19.97 | 2.77 | 11.63 |
| | Multi-layer | 19.05 | 17.32 | 2.57 | 9.18 |
| 7 and 8 | Tissue Only | 14.60 | 8.80 | 0.24 | 0.42 |
| | Multi-layer | 15.53 | 9.90 | 0.23 | 1.19 |

Significant visual contrast values were achieved with a liner used as the top sheet of the system. Unexpectedly, the liner only showed a small reduction in visual contrast. For example, Samples 1 and 2 (compared) retained 95% of the brightness visual contrast when the liner was placed on top. Samples 3 and 4 (compared) retained 88% of the brightness visual contrast when the liner was placed on top. Samples 5 and 6 (compared) retained 90% of the brightness visual contrast when the liner was placed on top. Samples 7 and 8 (compared) actually had an increase of 6% in the brightness visual contrast when the liner was placed on top. Therefore, the invention can be effective in providing visual information even when placed below a material such as the liner.

Participants in a series of consumer focus groups were shown various visual patterns placed in diapers and asked for their reaction. The concept samples simulating wet diapers were made by inserting printed containment tissue into fully functional diaper shells. The untreated hydrophilic (wet) areas were simulated with a light gray tone matching the contrast differences achieved by the invention. Numerous patterns including those disclosed here as well as several new designs (swirls and circles) were evaluated.

The concept samples were presented to each group of panelists on a table with instructions only to visual examine each one without handling. Unaided reaction was first recorded. Further questions were directed at how well specific functions for the tissue (integrity, wetness, skin health, softness and aesthetics) were communicated by the concept. Unaided, the swirls and circles were largely interpreted functionally as “wetness” indicators. Geometric-type patterns (grid and cheese cloth) were best at communicating improved integrity. Further discussions with the groups indicated that designs with fine elements were preferred. Larger element designs were less aesthetically appealing.

Wet Permeability Testing

Wet permeability testing was performed on Samples 1 and 7 to demonstrate that treated regions of this invention can increase air flow through a containment sheets, a prerequisite for the improved breathability. Sample properties are described above in Table 1. The hydrophobic and hydrophilic tissues were originated from the same base material to minimize variability. Samples 1 and 7 were tested by printing two patterns, a “fine grid” pattern (Samples 1.1 and 7.1) and a “course grid” pattern (Samples 1.2 and 7.2), of sizing agent treated regions onto sample sheets. For comparison, Samples 1 and 7 were also tested without any treatment as a control.

The hydrophobic treatment of Sample 1.1 was achieved with Hercon 70 sizing chemistry applied by gravure printing using a 7.0 cbm/in² pattern and a flexographic plate with a “fine grid” pattern. The hydrophobic treatment of Sample 1.2 was achieved with Hercon 70 sizing chemistry applied by gravure printing using a 7.0 cbm/in² pattern and flexographic plate with a “coarse grid” pattern as shown generally in FIG. 6. The hydrophobic treatments of Sample 7.1 was achieved with Hercon 70 sizing chemistry applied by gravure printing using a 4.0 cbm/in² pattern and a flexographic plate with the “fine grid” pattern. The hydrophobic treatments of Sample 7.2 was achieved with Hercon 70 sizing chemistry applied by gravure printing using a 4.0 cbm/in² pattern and a flexographic plate with the “course grid” pattern. The fine and course grid patterns resembled the grid pattern of FIG. 5. The fine grid pattern comprised hydrophilic squares of about 4 mm×4 mm divided by an about 0.5 mm line of sizing agent. The course grid pattern comprised hydrophilic squares of about 10 mm×10 mm divided by an about 2.0 mm line of sizing agent.

The “wetted air permeability” of the samples was measured using a Dynamic Drainage Analyzer (DDA) (available from AB Akribi Kemikonsulter, Sundsvall, Sweden). Set-up, calibration and operating procedures were followed according to the Instruction Manual (version WP2.x, November 1999). The instrument is designed to give wet sheet permeability, the property of interest for characterizing the invention. The apparatus consists of a drainage unit, control systems for electronic and pneumatic systems, a computer for data acquisition, and application software. The drainage unit consists of a vacuum vessel connected to a vacuum pump and an adjustable bleed valve and vacuum gauge. A special wire and gasket assembly is used to support the sample.

All experiments were run with the vacuum set and calibrated to 0.2 bar. Vacuum was set by starting the vacuum pump and turning the screw on the bleed valve on top of the vacuum vessel until the vacuum read 0.2 millibar on the pressure gauge attached to the vacuum vessel. The correct value was then entered into the program. The test time was set to 20 seconds. This provided adequate time for excess water to be removed and the vacuum to level out. The “wetted air permeability” value was taken at 15 seconds after the test was started. In all cases, the vacuum trace leveled off well ahead of this point.

Normal operation of the DDA sample uses a formed or fitted sample that completed covers the full face of the wire screen. For the relatively high air permeabilities of the tissue being evaluated, the effective open area had to be decreased. Therefore, an impermeable conformable natural latex rubber sheet with a 25 mm diameter opening was used to narrow the effective opening in which to draw the vacuum through the wire. The outer diameter of the latex gasket was large enough to extend and clamp underneath the removable top cylinder of the apparatus thus making a complete seal except for the 25 mm diameter opening. The latex rubber sheet is of the same material used in test methods to determine saturation capacity.

Each sample was prepared for testing by die-cutting a 34 mm diameter single ply sheet from the sample material. Care was taken to insure that it had no wrinkles, tears, or defects that could create additional openings for air to pass unimpeded around the sample.

Typically, the spring/Teflon gasket was left on the piston housing as well as the metal ring place on top of it from one test to another. Next, the wire was positioned in the drainage apparatus as specified in the operating instructions. The dry specimen was then positioned in the center of the supporting wire. The rubber gasket was then positioned over the sample and with the opening in the gasket centered on the sample. The metal support with an O-ring gasket was then placed over the gasket material. Finally, the cylinder housing was aligned and clamped into place. Visual inspection provided that the gasket overlapped the specimen, lay smoothly across the wire, and was securely clamped around the outer edge by the cylinder and ring. Between tests, it is important to insure that all surfaces were dried to avoid accidental wetting of the samples prior to testing. The appropriate identifying and file information was entered into the INPUT screen.

For dry permeability, the DDA test program is then started. The DDA vacuum is taken at a reasonably level portion of the pressure trace. The data here was taken at the 15 second mark (after the test was started). The data is given in bars and multiplied by 1000 to convert to millibars pressure. Three tests were done for each sample and the average is reported in Table 10 for both dry and wet permeability.

For wet permeability, a volume of 75 ml of distilled water (equilibrated to 20° C.) was placed in a 100-ml beaker. The DDA test program is then started. Within five seconds of starting the apparatus, the entire volume of water was poured down the wall of the cylinder so that it washed over the dry specimen. The rate of pouring was sufficient to completely cover the specimen with a layer of water and achieve wetting of all hydrophilic areas. This causes a rapid initial spike in vacuum followed by a rapid drop off. The pressure traces indicate a maximum of three seconds from the time of the first initial rise in vacuum to the inflection indicating the “dryline”. The DDA vacuum is taken at a reasonably level portion of the pressure trace after the “dryline” is achieved. The data here was taken at the 15-second mark (after the test was started), which was typically five to 10 seconds after the appearance of the “dryline.” The data is given in bars and multiplied by 1000 to convert to millibars pressure. Three tests were done for each code and the average is reported in Table 10 for both dry and wet permeability.

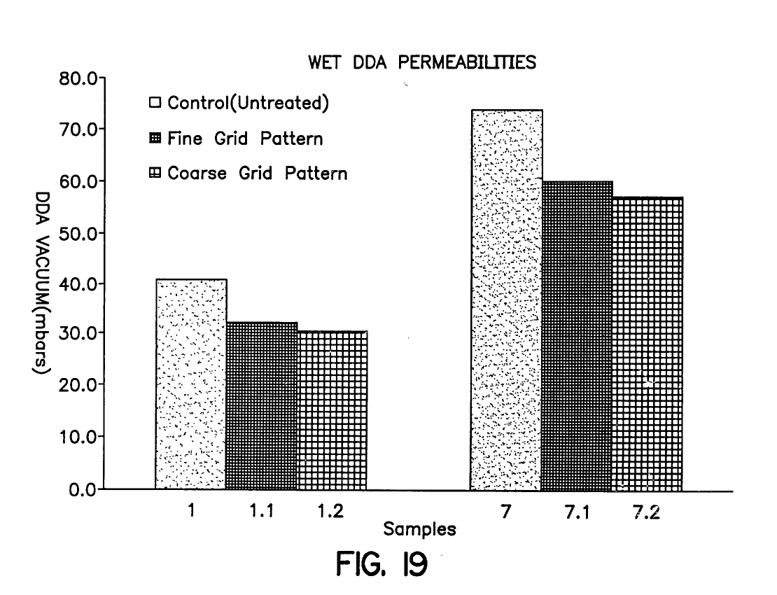

Test results for the Samples are summarized in Table 10. Wet permeability is reported directly as “DDA Vacuum” in units of millibars. A lower value represents higher air permeability. FIG. 19 summarizes the results of Table 10 and shows that both Samples 1.1, 1.2, 7.1, and 7.2, tissues having either a fine grid or coarse grid hydrophobic patterns, have significantly better wet permeability than the untreated control. For Sample 1, a wet permeability improvement of 21% for the fine grid patterns and 25% for the coarse grid patterns was achieved. For Sample 7, a wet permeability improvement of 19% for the fine grid pattern and 22% for the coarse grid pattern was achieved.

TABLE 10

| | Flexo Plate & | DDA Dry | DDA Wet |
|---|---|---|---|
| | Chemical | Permeability | Permeability |
| Sample | Treatment | (mbar) | (mbar) |
| 1 | None (control) | 6.3 | 41.0 |
| 1.1 | Fine Grid, Hercon 70 | 7.0 | 32.3 |
| 1.2 | Coarse Grid, Hercon 70 | 6.3 | 30.7 |
| 7 | None (control) | 17.7 | 74.0 |
| 7.1 | Fine Grid, Hercon 70 | 20.3 | 60.3 |
| 7.2 | Coarse Grid, Hercon 70 | 21.7 | 57.3 |

Tensile Testing

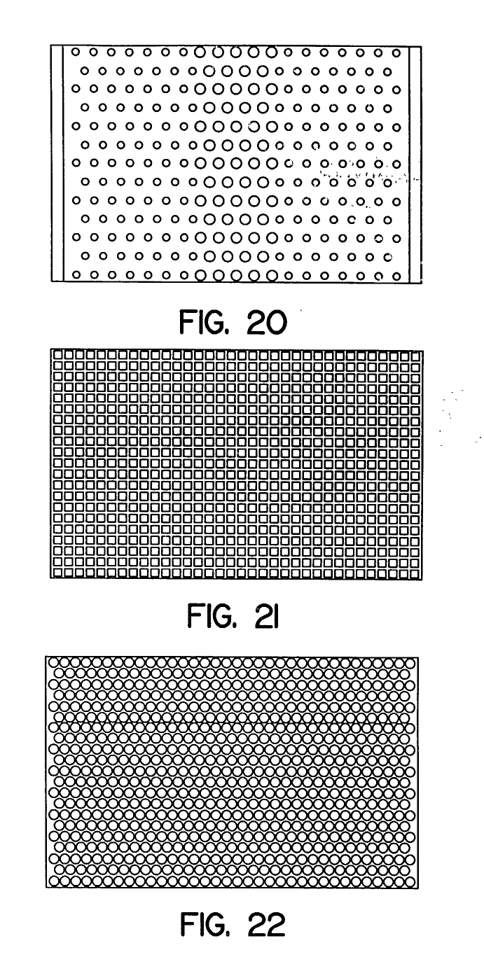

To demonstrate the use of this invention in improving the wet strength of a containment tissue a tensile test was done by the method below. Samples 9-12 were prepared for the tensile testing. Samples 9-12 were made from an UCTAD white containment tissue made with relatively low z-direction topography transfer and TAD fabrics containing 50% northern softwood kraft and 50% softwood BCTMP. Sample 9 was a control sample and was not treated with any hydrophobic agents of this invention, and was therefore hydrophilic. Samples 10-12 were comprised the UCTAD base tissue of Sample 9 and all had additional treatment. Sample 10 was printed using a magnetic flexographic 425 mm wide plate having a configuration as shown in FIG. 20, with the larger center circles having a 14 mm diameter and the smaller circles having a 10 mm diameter. The circles represent the raised portion of the plate. Sample 11 was printed using a magnetic flexographic 425 mm wide plate having a configuration as shown in FIG. 21, with each square having a 2 mm×2 mm dimension. The shaded grid area represents the raised portion of the plate. Sample 12 was printed using a magnetic flexographic 425 mm wide plate having a configuration as shown in FIG. 22, with each circle having a 10 mm diameter. The shaded grid area represents the raised portion of the plate.

The printing of Samples 10-12 was done using a Precis 2000 sizing agent having a 26% solid measurement. The flexographic printing of Samples 10-12 was done using a variation of the pilot line configuration of FIG. 9. A 7.0 cbm gravure printing roll was used for gravure roll 60 (lower) and no upper gravure roll 60 was present. A magnetic roll was substituted for intermediate roll 64. The appropriate flexographic rubber printing plates, as discussed above, were magnetically attached to the magnetic roll. Light pre-calendering was done on the samples byrolls 58. The add-on percent for Samples 10-12 was approximately 1.9% for the printed regions. From the printed samples both machine direction (MD) and cross-direction (CD) strips were cut for the appropriate tensile testing.

The dry and wet machine direction tensile strength, machine direction tensile stretch, cross-direction tensile strength and cross-direction tensile stretch are obtained according to TAPPI Test Method 494 OM-88 “Tensile Breaking Properties of Paper and Paperboard,” herein incorporated by reference, using the following parameters: crosshead speed is 10.0 in/min. (254 mm/min), full scale load is 10 lb (4,540 g),jaw span (the distance between the jaws, sometimes referred to as the gauge length) is 4.0 inches (101.6 mm), sample width is 3 inches (76.2 mm). All tensile tests were performed using a 1-ply sample. Wet strength tensile testing was performed by wetting the center of the test strip with a clean wetted sponge using distilled water: A sponge width of either 15 mm or 22 mm was selected so that an approximate equal wetted profile was achieved for the sample set being compared. Samples with closed zones of hydrophilicity (as in a grid pattern) used a 22 mm wide sponge. Samples with contiguous regions of hydrophilicity in the direction being tested used a 15 mm sponge. The tensile testing machine was a Sintech, Model CUTS1000 (Systems Integration Technology Inc., Stoughton, Mass.; a division of MTS Systems Corporation, Research Triangle Park, N.C.).

Machine direction and cross-direction dry and wet tensile tests were completed for Samples 9-12. Ten repetitions were done for each sample and the results are averaged and summarized along with the standard deviation in Table 11. For all printed patterns, the machine direction wet tensile strength (709 vs. 641) and cross-direction wet tensile strength (520 vs. 449) was significantly higher than that of the control Sample 9. In addition, the wet strength efficiency, “% Wet/Dry” (wet tensile strength/dry tensile strength×100) was also increased over the control. For machine direction % Wet/Dry the average ratio for printed tissue was 55.5% compared to 43.69 for the control. For cross-direction % Wet/Dry, the average ratio for the printed tissues was 47.8% compared to 36.3% for the control.

TABLE 11

| | | | | % MD | | | % CD |
|---|---|---|---|---|---|---|---|
| | | MD Dry | MD Wet | Wet/Dry | CD Dry | CD Wet | Wet/Dry |
| Sample | | (g/3-inch) | (g/3-inch) | (%) | (g/3-inch) | (g/3-inch) | (%) |
| 9 | Ave. | 1364 | 724 | 53.1 | 1159 | 518 | 44.7 |
| | St. Dev. | 49 | 50 | | | 13 | |
| 10 | Ave. | 1253 | 704 | 56.2 | 1116 | 526 | 47.1 |
| | St. Dev. | 34 | 36 | | 17 | 33 | |
| 11 | Ave. | 1225 | 700 | 57.1 | 1082 | 517 | 47.8 |
| | St. Dev. | 55 | 33 | | 37 | 42 | |
| 12 | Ave. | 1470 | 641 | 43.6 | 1237 | 449 | 36.3 |
| | St. Dev. | 59 | 27 | | 26 | 8 | |

While the embodiments of the invention described herein are presently preferred, various modifications and improvements can be made without departing from the spirit and scope of the invention. The scope of the invention is indicated by the appended claims, and all changes that fall within the meaning and range of equivalents are intended to be embraced therein.

We claim:

1. A containment tissue, comprising:
a fiber matrix; and
at least one treated region comprising a hydrophobic agent applied on the fiber matrix in a predetermined pattern.

2. The containment tissue of claim 1, comprising at least one uncreped through air dried tissue ply.

3. The containment tissue of claim 2, wherein the at least one treated region further comprises a percent rewet of less than about 20% at an applied pressure of 1.0 psi.

4. The containment tissue of claim 3, wherein the at least one treated region further comprises a percent rewet of less than about 15% at an applied pressure of 1.0 psi.

5. The containment tissue of claim 4, wherein the at least one treated region further comprises a percent rewet of less than about 10% at an applied pressure of 1.0 psi.

6. The containment tissue of claim 1, comprising at least one creped Yankee dried tissue ply.

7. The containment tissue of claim 6, wherein the at least one treated region further comprises a percent rewet of less than about 60% at an applied pressure of 1.0 psi

8. The containment tissue of claim 7, wherein the at least one treated region further comprises a percent rewet of less than about 40% at an applied pressure of 1.0 psi.

9. The containment tissue of claim 8, wherein the at least one treated region further comprises a percent rewet of less than about 25% at an applied pressure of 1.0 psi.

10. The containment tissue of claim 1, wherein the at least one treated region further comprises a dryness improvement percent of at least about 25% at an applied pressure of 1.0 psi.

11. The containment tissue of claim 10, wherein the at least one treated region further comprises a dryness improvement percent of at least about 50% at an applied pressure of 1.0 psi.

12. The containment tissue of claim 1, wherein the hydrophobic agent is applied by at least one of printing, spraying, and inkjet printing.

13. The containment tissue of claim 1, wherein the hydrophobic agent comprises a compound selected from the group consisting of alkyl ketene dimers, alkenyl succinic anhydrides, latex compounds, hydrophobic silicone compounds, and combinations thereof.

14. The containment tissue of claim 1, wherein the fiber matrix further comprises synthetic fibers.

15. The containment tissue of claim 1, wherein the at least one treated region has a hydrostatic head value of at least about 5 millibars.

16. The containment tissue of claim 15, wherein the at least one treated region has a hydrostatic head value of at least about 10 millibars.

17. The containment tissue of claim 16, wherein the at least one treated region has a hydrostatic head value of at least about 16 millibars.

18. The containment tissue of claim 1, wherein the at least one treated region has an air permeability value of at least about 25 cfm/min/ft².

19. The containment tissue of claim 18, wherein the at least one treated region has an air permeability value of at least about 60 cfm/min/ft².

20. The containment tissue of claim 19, wherein the at least one treated region has an air permeability value of at least about 150 cfm/min/ft².

21. The containment tissue of claim 1, wherein the at least one treated region has a % Wet/Dry tensile strength of at least about 15%.

22. The containment tissue of claim 21, wherein the at least one treated region has a % Wet/Dry tensile strength of at least about 30%.

23. The containment tissue of claim 1, wherein the at least one treated region has a sizing agent percent add-on of at least about 0.1%.

24. The containment tissue of claim 23, wherein the at least one treated region has a sizing agent percent add-on of at least about 0.5%.

25. The containment tissue of claim 24, wherein the at least one treated region has a sizing agent percent add-on of at least about 1.0%.

26. The containment tissue of claim 1, further comprising at least two treated regions separated by at least one untreated region.

27. The containment tissue of claim 26, wherein each treated region comprises a size of at least about 1 square millimeter.

28. The containment tissue of claim 27, wherein each treated region comprises a size of at least about 10 square millimeters.

29. The containment tissue of claim 28, wherein each treated region comprises a size of at least about 75 square millimeters.

30. The containment tissue of claim 29, wherein each treated region comprises a size of at least about 150 square millimeters.

31. The containment tissue of claim 1, further comprising at least two treated regions wherein at least one of the at least two treated is a different size.

32. The containment tissue of claim 1, further comprising at least two untreated regions separated by at least one treated region.

33. The containment tissue of claim 32, wherein each untreated region comprises a size of at least about 1 square millimeter.

34. The containment tissue of claim 33, wherein each untreated region comprises a size of at least about 10 square millimeters.

35. The containment tissue of claim 34, wherein each untreated region comprises a size of at least about 75 square millimeters.

36. The containment tissue of claim 35, wherein each untreated region comprises a size of at least about 150 square millimeters.

37. The containment tissue of claim 1, further comprising at least two untreated regions wherein at least one of the at least two untreated is a different size.

38. The containment tissue of claim 1, further comprising a density of less than about 0.25 grams per cubic centimeter.

39. The containment tissue of claim 38, further comprising a density of less than about 0.15 grams per cubic centimeter.

40. The containment tissue of claim 39, further comprising a density of less than about 0.10 grams per cubic centimeter.

41. An absorbent article, comprising:
a liquid-permeable body-side liner;
a containment tissue adjacent to the body-side liner comprising a fiber matrix and at least one treated region wherein a hydrophobic agent is applied to the fiber matrix in the fiber matrix in the at least one treated region;
an absorbent core adjacent the containment tissue; and
a substantially liquid-impermeable outer cover adjacent to the absorbent core.

42. The absorbent article of claim 41, wherein at least one treated region comprises a predetermined pattern.

43. The absorbent article of claim 41, wherein the containment tissue further comprises at least one uncreped through air dried tissue ply.

44. The absorbent article of claim 43, wherein the at least one treated region further comprises a percent rewet of less than about 20% at an applied pressure of 1.0 psi.

45. The absorbent article of claim 44, wherein the at least one treated region further comprises a percent rewet of less than about 15% at an applied pressure of 1.0 psi.

46. The absorbent article of claim 45, wherein the at least one treated region further comprises a percent rewet of less than about 10% at an applied pressure of 1.0 psi.

47. The absorbent article of claim 41, wherein the containment tissue comprising at least one creped Yankee dried tissue ply.

48. The absorbent article of claim 47, wherein the at least one treated region further comprises a percent rewet of less than about 60% at an applied pressure of 1.0 psi

49. The absorbent article of claim 48, wherein the at least one treated region further comprises a percent rewet of less than about 40% at an applied pressure of 1.0 psi.

50. The absorbent article of claim 49, wherein the at least one treated region further comprises a percent rewet of less than about 25% at an applied pressure of 1.0 psi.

51. The absorbent article of claim 41, wherein the at least one treated region further comprises a dryness improvement percent of at least about 25% at an applied pressure of 1.0 psi.

52. The absorbent article of claim 51, wherein the at least one treated region further comprises a dryness improvement percent of at least about 50% at an applied pressure of 1.0 psi.

53. The absorbent article of claim 41, wherein the hydrophobic agent is applied by at least one of printing, spraying, and inkjet printing.

54. The absorbent article of claim 41, wherein the hydrophobic agent comprises a compound selected from the group consisting of alkyl ketene dimers, alkenyl succinic anhydrides, latex compounds, hydrophobic silicone compounds, and combinations thereof.

55. The absorbent article of claim 41, wherein the fiber matrix comprises wood pulp fibers.

56. The absorbent article of claim 55, wherein the fiber matrix further comprises synthetic fibers.

57. The absorbent article of claim 41, wherein the at least one treated region has a hydrostatic head value of at least about 5 millibars.

58. The absorbent article of claim 57, wherein the at least one treated region has a hydrostatic head value of at least about 10 millibars.

59. The absorbent article of claim 58, wherein the at least one treated region has a hydrostatic head value of at least about 16 millibars.

60. The absorbent article of claim 59, wherein the at least one treated region has an air permeability value of at least about 25 cfm/min/ft².

61. The absorbent article of claim 60, wherein the at least one treated region has an air permeability value of at least about 60 cfm/min/ft².

62. The absorbent article of claim 61, wherein the at least one treated region has an air permeability value of at least about 150 cfm/min/ft².

63. The absorbent article of claim 41, wherein the at least one treated region has a % Wet/Dry tensile strength of at least about 15%.

64. The absorbent article of claim 63, wherein the at least one treated region has a % Wet/Dry tensile strength of at least about 30%.

65. The absorbent article of claim 41, wherein the at least one treated region has a sizing agent percent add-on of at least about 0.1%.

66. The absorbent article of claim 65, wherein the at least one treated region has a sizing agent percent add-on of at least about 0.5%.

67. The containment tissue of claim 66, wherein the at least one treated region has a sizing agent percent add-on of at least about 1.0%.

68. The absorbent article of claim 41, wherein the absorbent article comprises a diaper.

69. The absorbent article of claim 41, wherein the absorbent article comprises a swimpant.

70. The absorbent article of claim 41, wherein the absorbent article comprises a training pant.

71. The absorbent article of claim 41, wherein the absorbent article comprises a feminine care product.

72. The absorbent article of claim 41, wherein the absorbent article comprises a medical absorbent product.

73. An absorbent article, comprising:
a liquid-permeable body-side liner;
a containment tissue adjacent to the body-side liner and wrapped around an absorbent core; and
a substantially liquid-impermeable outer cover adjacent to the wrapped absorbent core;
wherein the containment tissue comprises a fiber matrix including at least one treated region, and at least one sizing agent applied in the at least one treated region.

74. The absorbent article of claim 73, wherein the at least one treated region covers substantially all of a surface of the absorbent core facing the outer cover.

75. The absorbent article of claim 74, wherein the at least one treated region covers sides of the absorbent core.

76. The absorbent article of claim 73, wherein the containment tissue further comprises at least one uncreped through air dried tissue ply.

77. The absorbent article of claim 76, wherein the at least one treated region further comprises a percent rewet of less than about 20% at an applied pressure of 1.0 psi.

78. The absorbent article of claim 77, wherein the at least one treated region further comprises a percent rewet of less than about 15% at an applied pressure of 1.0 psi.

79. The absorbent article of claim 78, wherein the at least one treated region further comprises a percent rewet of less than about 10% at an applied pressure of 1.0 psi.

80. The absorbent article of claim 73, wherein the containment tissue comprising at least one creped Yankee dried tissue ply.

81. The absorbent article of claim 80, wherein the at least one treated region further comprises a percent rewet of less than about 60% at an applied pressure of 1.0 psi

82. The absorbent article of claim 81, wherein the at least one treated region further comprises a percent rewet of less than about 40% at an applied pressure of 1.0 psi.

83. The absorbent article of claim 82, wherein the at least one treated region further comprises a percent rewet of less than about 25% at an applied pressure of 1.0 psi.

84. The absorbent article of claim 73, wherein the at least one treated region further comprises a dryness improvement percent of at least about 25% at an applied pressure of 1.0 psi.

85. The absorbent article of claim 84, wherein the at least one treated region further comprises a dryness improvement percent of at least about 50% at an applied pressure of 1.0 psi.

86. The absorbent article of claim 73, wherein the hydrophobic agent is applied by at least one of printing, spraying, and inkjet printing.

87. The absorbent article of claim 73, wherein the hydrophobic agent comprises a compound selected from the group consisting of alkyl ketene dimers, alkenyl succinic anhydrides, latex compounds, hydrophobic silicone compounds, and combinations thereof.

88. The absorbent article of claim 73, wherein the absorbent article comprises a diaper.

89. The absorbent article of claim 73, wherein the absorbent article comprises a feminine care product.
